(19) United States
(12) Patent Application Publication
(10) Pub. No.: US 2026/0100276 A1
(43) Pub. Date: Apr. 9, 2026

(54) SYSTEMS AND METHODS FOR CLINICAL DATA EXCHANGE FROM ELECTRONIC HEALTH RECORD SYSTEMS TO PARTICIPANTS

(71) Applicant: Oracle International Corporation, Redwood Shores, CA (US)

(72) Inventors: Jesus Velazquez Reyes, Seattle, WA (US); Josh Diaz, Kansas City, MO (US); Siva Edupuganti, Seattle, WA (US); Santosh Shilimkar, Belmont, CA (US); Nemanja Dordevic, Brooklyn, NY (US); Tadesse Feyissa, Auburn, WA (US); Bhuvan Damodhar, Kansas City, MO (US); Girish Chawla, Malvern, PA (US)

(73) Assignee: Oracle International Corporation, Redwood Shores, CA (US)

(21) Appl. No.: 19/282,848

(22) Filed: Jul. 28, 2025

Related U.S. Application Data
(60) Provisional application No. 63/705,462, filed on Oct. 9, 2024.
(60) Provisional application No. 63/713,459, filed on Oct. 29, 2024.

(51) Int. Cl. G16H 40/67; G16H 10/60

(52) U.S. Cl. CPC G16H 40/67; G16H 10/60

(57) ABSTRACT

The present disclosure relates to cloud-based centralized clinical data exchange (CDeX) techniques leveraging a unified interoperability interface for sharing selectively and/or dynamically medical records of subjects with other participants. In some aspects, techniques may be provided to facilitate, support or perform notifying participants or external entities (e.g., regulators, payers, insurance companies or other entities) in real-time or near real-time when a subject encounter occurs and/or throughout the encounter by transmitting admission, discharge, and transfer (ADT) messages. The unified interoperability interface may enable data sharing by establishing subject-specific and/or participant-specific communication channels that can be initiated by either party, provided that the participants are on-boarded and registered within the CDeX system.

### CROSS-REFERENCE TO RELATED APPLICATIONS

This application claims the benefit of and the priority to U.S. Provisional Application No. 63/705,462, filed on Oct. 9, 2024, and U.S. Provisional Application No. 63/713,459, filed on Oct. 29, 2024. Each of these applications is hereby incorporated by reference in its entirety for all purposes.

### BACKGROUND

Healthcare organizations (HCOs) are increasingly required to share subject medical records with a diverse range of participants, including state agencies, insurance companies, and other healthcare providers. This information may be used for various operational functions such as pre-authorization, risk adjustment, and the calculation of Healthcare Effectiveness Data and Information Set (HEDIS) measures. However, the transactional models utilized for information exchange may often be fragmented, involving methods such as mail, fax, web portals, and point-to-point ad-hoc communications. This complexity may not only incur significant operational costs but also necessitate the involvement of third-party vendors, which can introduce substantial delays in obtaining information. The reliance on multiple solutions and communities may complicate the on-boarding process (i.e., integrating new participants), creating inefficiencies in administrative operations, approvals, and regulatory reviews.

Additionally, clinical data exchange may emphasis on privacy and regulatory compliance, as healthcare providers often follow specific state and federal guidelines that dictate how sensitive personal information is handled, including circumstances under which such information must be withheld. Furthermore, the permissions for various subjects may differ significantly based on their roles and the type of information they require, complicating the exchange process. Apart from this, current solutions in the healthcare landscape may often fall short in facilitating timely and proactive information sharing. For instance, insurance companies frequently lack real-time visibility into clinical events occurring within HCOs, such as subject admissions. Without a general notification mechanism, insurers remain unaware of significant subject developments until the healthcare organization initiates contact for pre-authorization of procedures. This reactive approach can hinder patient care and create bottlenecks in the administrative workflow, as payers may require additional information only after a procedure has been planned. Addressing these communication gaps may be a concern for enhancing collaboration between participants including healthcare providers and external entities, leading to improved patient outcomes and streamlined operations.

### SUMMARY

Certain aspects and features of the present disclosure relate to cloud-based centralized clinical data exchange (CDeX) techniques leveraging a unified interoperability interface for sharing selectively and/or dynamically medical records of subjects with other participants. In some aspects, techniques may be provided to facilitate, support or perform notifying participants or external entities (e.g., regulators, payers, insurance companies or other entities) in real-time or near real-time when a subject encounter occurs and/or throughout the encounter by transmitting admission, discharge, and transfer (ADT) messages. The real-time ADT notification mechanism may include detecting an event trigger related to an initiation of the subject encounter in a healthcare provider domain. The healthcare provider domain may include one or more healthcare providers, each leveraging a millennium platform e.g., electronic health records (EHRs) to enhance clinical operations, streamline data management, and improve patient care coordination across the continuum of services. Each millennium platform may include an interface (e.g., an open interface including open APIs and open standards such as HTTP), serving as a gateway for handling (i.e., sending and/or receiving) ADT messages.

Upon detection of the event trigger, one or more ADT messages (or ADT feed) associated with the event trigger may be generated in real-time via the open interface associated with a healthcare provider, configured to transmit the ADT messages to a backend cell. The ADT messages may include notifications associated with one or more of subject encounters including: admission, transfer, discharge, visit, end visit, register, pre-admission and update information. In some examples, the ADT messages may comply with HL7 standard.

The backend cell, configured within a cloud service, may receive the ADT messages from the open interface in real-time and forward these to a frontend cell after processing. The backend cell may issue an acknowledgment (ACK) to confirm that an ADT message has been successfully received and validated based on its compliance with predefined rules, including adherence to specific protocols such as the HL7 standard. In instances where issues occur—such as incorrect data formats, invalid data types, or missing fields—a negative acknowledgment (NACK) message may be generated, detailing the specific problem encountered. Additionally, the backend cell may implement a load balancer to distribute ADT messages across various application fleets (i.e., applications or microservices that work together to deliver a particular functionality or service), thereby enabling the disclosed CDeX system to scale efficiently and accommodate more healthcare providers while effectively managing incoming traffic.

The frontend cell, a component of the CDeX data plane, may process the ADT messages by assigning a master subject index that uniquely identifies each subject within the healthcare provider domain. This method may facilitate the creation of a global subject directory, aggregating medical information and ADT messages generated during specific subject encounters, thus allowing for efficient searches across the healthcare provider domain using a unique identifier (ID). Following processing, the frontend cell may identify one or more external entities from the ADT messages based on a set of identifiers (e.g., tenancy ID, organization ID) assigned by the CDeX system during the onboarding or registration of these external entities.

In some aspects, the ADT messages associated with the subject encounter may be stored in association with the master subject index, along with the set of identifiers to record an encounter lifecycle including the subject encounter and one or more related subject encounters. For example, when a subject (e.g., a patient) is admitted to a hospital, an ADT message may be generated. Throughout the subject's stay, subsequent real-time ADT messages may be generated document various encounters. Finally, upon discharge, an ADT message may be sent to signify the completion of the encounter, encapsulating the discharge instructions, billing information, follow-up appointments, and any necessary referrals, thereby creating a complete lifecycle of the subject encounter within the healthcare provider domain. Additionally, techniques may be provided in which the external entity may request via the interoperability interface to display the encounter lifecycle. In response to the request, the encounter life cycle including the particular subject encounter may be shown in a graphical user interface to the external entity.

In some other aspects, some techniques disclosed herein provide users (e.g., healthcare providers) with the ability to tailor the data-sharing process. To this end, ADT feed or messages may be dynamically selected or filtered, via the frontend cell, based on data-sharing permissions that are configured by the healthcare provider for the one or more external entities. The selected or filtered ADT messages may be transmitted to each of the identified external entities via an interoperability interface configured within the frontend cell, thereby enabling real-time ADT feed received by the external entities.

In certain instances, the unified interoperability interface may facilitate seamless clinical data exchange among various participants or external entities, regardless of the underlying data structure. This interface may enable data sharing by establishing subject-specific communication channels that can be initiated by either party, provided that the participants are on-boarded and registered within the CDeX system. For example, an external entity may initiate a request to access clinical information related to a subject encounter by creating a client application within its designated client identity domain. This process may involve inputting relevant identifiers associated with both the client identity domain and the client application through a console provided by the CDeX system. During creation, the client application may request an access token from the client identity domain. Based on the identifiers, the access token may be issued to authenticate the external entity, granting it permission to initiate the request.

Once the token is granted, one or more requests—including the access token—may be transmitted from the client application via a query endpoint associated with the interoperability interface. In some examples, the external entity provides a service uniform resource locator (URL) as the query endpoint. This query endpoint may be configured to send requests for clinical information associated with the particular subject encounter from the healthcare provider domain (or a specific healthcare provider), through the console. Additionally, the query endpoint may be configured to receive the one or more requests concurrently from the external entity, with request rate limits applied via an API gateway.

For receiving, analogous to query endpoint, a delivery endpoint may be created for receiving the clinical information in response to the request. After the creation of endpoints, a communication channel may be established between the healthcare provider domain and the external entity by linking the query endpoints to the delivery endpoints. Using the communication channel, one or more requests may be received by the engine configured within the cloud service that may process the request to identify the subject and a healthcare provider associated with the subject encounter holding the clinical information based on a master subject index (MSI) that uniquely identifies a subject across a healthcare provider domain. The engine may further apply subject-specific data-sharing permissions and data governance rules to determine whether the clinical information is authorized to share with the external entity. This approach may enable dynamic and selective data access improving privacy and regulatory compliance.

Upon authorization, the clinical information may be retrieved by the engine from the identified healthcare provider. A response may be generated including the clinical information in a standardized format that may be transmitted to the external entity via the established communication channel. In some examples, the established communication channel may be leveraged to update the external entity by transmitting real-time ADT feed (or messages) based on triggering subsequent subject encounters involving the identified healthcare provider. In some other examples, the clinical information may be cached temporarily at the engine to be accessed within a predetermined timeout period upon failure of fulfilling the requests.

The techniques may further include receiving an additional request from the external entity through the communication channel. The additional request may involve accessing further clinical information related to a specific encounter or modifications to the data-sharing permissions between the client and the healthcare provider. This capability may enable healthcare providers to dynamically adapt their data-sharing practices in real-time. For example, if a patient changes payers or if an external entity no longer requires access to certain data, the healthcare provider can promptly modify or revoke the relevant access permissions. Utilizing a control plane API, the configurations of components within the cloud service can be updated based on the additional request. In response, the engine may retrieve the master subject index according to the updated configurations, generating a subsequent response that includes a C62 bundle including the additional clinical information. This additionally generated response may be then transmitted to the external entity via the established communication channel.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows an exemplary block diagram to illustrate data flow between participants including a healthcare provider domain and external entities in accordance with some aspects of the present disclosure.

FIG. 2 illustrates an exemplary sequence diagram authenticating a client application associated with an external entity in accordance with some aspects of the present disclosure.

FIG. 3 illustrates a graphical representation of one aspect of selective data access depicting subject encounters over a specified date range.

FIG. 4A illustrates an exemplary graphical user interface (GUI) of the external entity, showing a healthcare provider directory.

FIG. 4B illustrates an exemplary GUI, showing a list of query endpoints from the healthcare provider directory of the FIG. 2.

FIG. 4C illustrates an exemplary GUI, providing a console for creating a client application for the external entity.

FIG. 5 shows an exemplary GUI, providing a console for creating a query endpoint for the external entity.

FIG. 6 shows an exemplary GUI, providing a console for creating a delivery endpoint for the external entity.

FIG. 7 illustrates an exemplary workflow for provisioning dynamic and selective data access between the healthcare provider domain and the external entities.

FIG. 8 illustrates an exemplary workflow for a unified interoperability interface enabling data sharing between the healthcare provider domain and the external entities.

FIG. 9 depicts a simplified diagram of a distributed system for implementing an embodiment of the present disclosure.

FIG. 10 is a simplified block diagram of a cloud-based system environment in accordance with certain aspects of various embodiments of the invention.

FIG. 11 illustrates an exemplary computer system that may be used to implement certain aspects.

### DETAILED DESCRIPTION

The present disclosure relates to cloud-based centralized clinical data exchange (CDeX) techniques for sharing selectively and/or dynamically medical records of subjects with other participants or external entities by leveraging a unified interoperability interface. Some techniques disclosed herein provide users (e.g., healthcare providers) with the ability to tailor the data-sharing process, deciding what specific clinical information to share with individual participants based on (for example) current and/or evolving subject circumstances, inter-party relationships, underlying requirements (e.g., legal requirements), etc. Alternatively, the disclosed techniques may provide the user, flexibility to define subject-specific and/or participant-specific data sharing permissions and data governance rules (as opposed to conventional all-or-nothing approaches), thereby improving privacy and regulatory compliance. By default, no clinical information is exchanged from healthcare providers to the external entity unless data-sharing permissions are configured.

Traditionally, external entities (e.g., regulators, payers, insurance companies or other entities) only receive information after the visit is completed, leading to delays in administrative processes, approvals, or regulatory reviews. In some aspects, techniques may be provided to facilitate, support or perform notifying participants or external entities in real-time or near real-time when a subject encounter occurs and/or throughout the encounter by transmitting admission, discharge, and transfer (ADT) messages. The disclosed techniques may integrate one or more electronic health record (EHR) systems or millennium platforms to send notifications either immediately (real-time) or within a short time frame (near real-time, within less than 5 minutes, within less than an hour, within less than a day, etc.) as the subject interaction begins. This quick responsiveness may allow external entities to initiate workflows with minimal lag, facilitating faster decisions and coordination.

The ADT messages may include notification including, admit, pre-admit or visit, discharge or end visit notification, registering a new subject, changing a status of the subject e.g., outpatient to inpatient, and/or updating information associated with a subject. An encounter in healthcare may refer to a specific interaction between a subject e.g., a patient and a healthcare provider during which services may be rendered and documented. For example, an admission encounter, a consultation encounter when visiting a medical specialist for evaluation of a specific condition, an emergency encounter e.g., seeking immediate care for an injury or acute illness, a discharge encounter when a subject is sent home after treatment, a transfer encounter for moving from the emergency department to the ICU (intense care unit), and a follow-up encounter for a post-operative check-up after surgery.

The real-time ADT notification mechanism may involve detecting an event trigger that marks the beginning of a subject encounter within a healthcare provider domain. This domain encompasses one or more healthcare providers, each utilizing a millennium platform—such as electronic health records (EHRs)—to enhance clinical operations, optimize data management, and improve patient care coordination throughout the continuum of services. Each millennium platform may feature an interface, such as an open interface with open APIs and standards like HTTP, which acts as a gateway for processing ADT messages, including both sending and receiving functionalities. Upon detecting the event trigger, the disclosed CDeX system may generate one or more ADT messages (or an ADT feed) in real time, utilizing the open interface associated with a healthcare provider. This interface may be set up to send the ADT messages to a backend cell. In some examples, these ADT messages may adhere to HL7 standards.

The disclosed CDeX system may be integrated into a cloud service, comprising a CDeX data plane designed to handle the processing and transmission, associated with ADT message and/or clinical data between services or components; and a CDeX control plane configured to manage and orchestrate the overall operations and policies. The CDeX data plane may further comprise a backend cell, responsible for receiving ADT messages from the open interface in real time, and a frontend cell to process the received ADT messages. The backend cell may issue an acknowledgment (ACK) to confirm the successful receipt and validation of an ADT message, enabling compliance with predefined rules, which may include adherence to specific protocols such as the HL7 standard. If any issues arise, such as incorrect data formats, invalid data types, or missing fields, a negative acknowledgment (NACK) message may be generated, outlining the specific problem encountered. Furthermore, the backend cell may incorporate a load balancer to distribute ADT messages across multiple application fleets (i.e., applications or microservices that collaboratively deliver a specific functionality or service). This implementation facilitates the efficient scaling of the CDeX system, allowing it to accommodate additional healthcare providers while effectively managing incoming traffic.

The ADT feed or messages associated with each healthcare provider may be sharded into smaller, more manageable segments to enhance storage efficiency and retrieval speed. The frontend cell may process these sharded ADT messages by assigning a master subject index (MSI) that uniquely identifies each subject within the healthcare provider domain. This approach may enable the establishment of a global subject directory that consolidates medical information and ADT messages generated during specific subject encounters, thereby facilitating efficient searches across the healthcare provider domain using a unique identifier (ID). After processing, the frontend cell may identify one or more external entities from the ADT messages based on a set of identifiers (e.g., tenancy ID, organization ID) assigned by the CDeX system during the onboarding or registration of these external entities. Additionally, a healthcare provider may be defined by a set of tenancy identifiers. Each ADT message may be extended to include the set of identifiers such as assigning authorities (AA), MSI, tenancy identifiers, medical record number (MRN), financial identification number (FIN), and/or an external entity identifier that corresponds to coverage for the subject.

In some aspects, the ADT messages associated with the subject encounter may be stored in association with the master subject index (MSI), along with the set of identifiers to record an encounter lifecycle including the subject encounter and one or more related subject encounters. For example, when a subject (e.g., a patient) is admitted to a hospital, an ADT message may be generated to capture admission details, including patient demographics, admission date, insurance plan, and the attending physician. Throughout the subject's stay, subsequent real-time ADT messages may be generated documenting transfers between departments, changes in care teams or healthcare providers, or updates in treatment plans. Upon discharge, an ADT message may be transmitted to indicate the conclusion of the encounter, encompassing discharge instructions, billing information, follow-up appointments, and any necessary referrals, thus creating a complete lifecycle of the subject encounter within the healthcare provider domain. Additionally, methods or techniques may be provided for the external entity to request the display of the encounter lifecycle through the interoperability interface. In response to this request, the encounter lifecycle, including the specific subject encounter, can be presented in a graphical user interface for the external entity.

In some other aspects, some techniques disclosed herein provide users (e.g., healthcare providers) with the ability to tailor the data-sharing process at a granular level, deciding what specific ADT messages or clinical information to share with individual participants (or external entities) based on (for example) current and/or evolving subject circumstances, inter-party relationships, underlying requirements (e.g., legal requirements), etc. The granularity may refer to subject-specific and/or participant-specific permissions. With this system, healthcare providers can configure these restrictions automatically, sharing only the necessary information with external entities such as auditors, government bodies, or payers. To this end, ADT feed or messages may be dynamically selected or filtered, via the frontend cell, based on data-sharing permissions and/or data governance rules that are configured by the healthcare provider for the one or more external entities. The selected or filtered ADT messages may be transmitted to each of the identified external entities via an interoperability interface configured within the frontend cell, thereby enabling real-time ADT feed received by the external entities.

By providing real-time or near real-time updates, external parties can process data pertaining to ongoing subject encounters dynamically, instead of passively waiting for encounter completion and/or post-encounter data. This can facilitate providing timely input from the external entity, to initiate early processing of related procedures. For example, it may be important for a regulator to monitor compliance with care protocols during an encounter, or it may be valuable (from a patient, provider, payer, etc. perspective) for an external entity to quickly assess the appropriateness or coverage of a procedure. Having access to live or nearly live data may enable these parties to actively engage in the process, which can improve the likelihood that appropriate approvals, compliance checks, or other tasks are addressed as the patient's care unfolds. Furthermore, this immediate or near-immediate notification capability may enhance internal workflows for these various entities. They can start executing tasks related to approvals, reviews, or audits based on live updates, improving operational efficiency and reducing delays in decision-making. This reduction in administrative lag not only accelerates the process but also minimizes disconnects, non-coverage, information unavailability, etc. that can arise when there is a significant time gap between patient care and data sharing.

In some embodiments, a unified interface can be provided that operates seamlessly across multiple EHR systems, enabling the exchange of clinical data regardless of the underlying infrastructure. Different healthcare providers often use distinct millennium (or EHR) platforms, each with its own standards, making it difficult for external entities to access and process subject data in a consistent format. Disclosed techniques may provide solutions to the problem by serving as a common interface that translates data from diverse EHR systems into a standard format, simplifying access for the external participants. An external entity may initiate a request for access to clinical information pertaining to a subject encounter by creating a client application within its assigned client identity domain. This process typically requires entering pertinent identifiers related to both the client identity domain and the client application via a console provided by the CDeX system. During this creation process, the client application may seek an access token from the client identity domain. The access token may be issued based on the identifiers, authenticating the external entity and permitting it to proceed with the request.

After the token is granted, one or more requests—including the access token—may be sent from the client application through a query endpoint linked to the interoperability interface. In some instances, the external entity may supply a service uniform resource locator (URL) to serve as the query endpoint. This query endpoint may be configured to send requests for clinical information related to the specific subject encounter from the healthcare provider domain (or a designated healthcare provider) via the console. Furthermore, the query endpoint can handle multiple requests simultaneously from the external entity, with request rate limits enforced through an API gateway.

Similar to the query endpoint, a delivery endpoint may be established for receiving the clinical information in response to the request. Once the endpoints are created, a communication channel may be established by linking the query endpoints to the delivery endpoints, facilitating interaction between the healthcare provider domain and the external entity. Through this communication channel, the engine may receive one or more requests and process them to identify the subject, and the healthcare provider associated with the subject encounter that holds the clinical information, utilizing the master subject index (MSI). Additionally, the engine may apply subject-specific data-sharing permissions and data governance rules to assess whether the clinical information can be shared with the external entity, thereby enabling dynamic and selective data access, enhancing privacy and regulatory compliance.

Upon receiving authorization, the engine can retrieve the clinical information from the identified healthcare provider. A response is then generated that includes the clinical information formatted according to established standards, which is transmitted to the external entity through the established communication channel. In some instances, this communication channel may also be utilized to keep the external entity updated by sending real-time ADT feeds (or messages) triggered by subsequent subject encounters with the identified healthcare provider. Additionally, in other examples, the clinical information may be temporarily cached at the engine for access within a predetermined timeout period in case of any request fulfillment failures.

The techniques may also involve receiving an additional request from the external entity through the communication channel. Th additional request may relate to accessing further clinical information related to a specific encounter or changes to the data-sharing permissions between the client and the healthcare provider. This functionality may enable healthcare providers to dynamically adjust their data-sharing practices in real-time. For instance, if a patient switches payers or if an external entity no longer needs access to specific data, the healthcare provider can quickly modify or revoke the corresponding access permissions. By utilizing a control plane API, the configurations of components within the cloud service can be updated based on this additional request. In response, the engine may retrieve the master subject index according to the revised configurations and generate a subsequent response that includes a C62 bundle containing the additional clinical information. This newly generated response can then be transmitted to the external entity via the established communication channel.

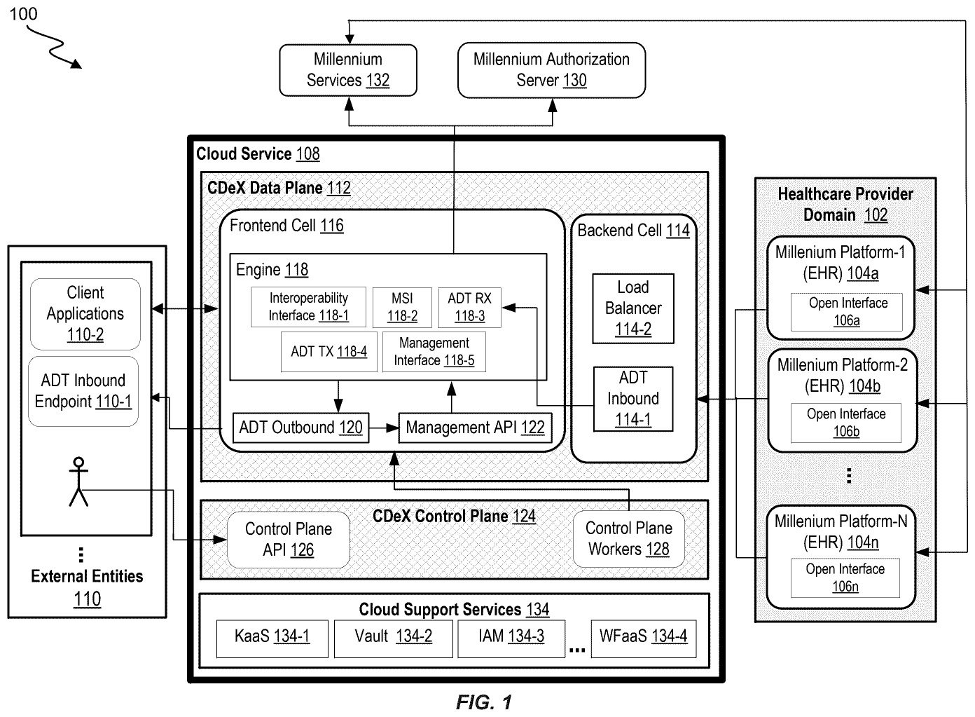

This interoperability may allow external parties to interact with healthcare data as if it came from a single, unified source, even though it may originate from multiple EHR platforms. The complexity of dealing with disparate systems is abstracted, allowing these entities to retrieve subject information, such as clinical notes or diagnostic test results, without considering which EHR system holds the data. For example, a regulatory body or payer could seamlessly request information regarding patient care and receive it in a standardized format, streamlining their review or approval processes. This standardized interface may also improve scalability and reduce operational costs. Instead of developing custom integrations with each EHR system individually, external participants can integrate with disclosed systems once and gain access to data from a wide range of healthcare providers. This may significantly reduce the technical and financial burdens of managing multiple connections and supports external parties easily accessing clinical data. Whether it is for regulatory oversight, payer approvals, or quality audits, these techniques provide a reliable and consistent data-sharing framework across the healthcare ecosystem FIG. 1 illustrates data flow through various components between healthcare provider domain 102 and one or more external entities 110, in accordance with some aspects of the present disclosure. The healthcare provider domain 102 may include one or more healthcare providers, each leveraging a millennium platform 104a, 104b, . . . , 104n e.g., electronic health records (EHRs). The millennium platform may hold clinical records of a healthcare provider, including details related to ADT events, to enhance clinical operations, streamline data management, and improve patient care coordination across the continuum of services. Each millennium platform may include an open interface 106a, 106b, . . . , 106n that may serve as a gateway for handling (i.e., sending and/or receiving) ADT (admission, discharge, and transfer) messages. These domains may include subject medical records including information related to ADT events. Each millennium domain may utilize its own open interface and a set of standards to enable seamless data exchange with EHRs and other healthcare technologies. The open interface 106 may be responsible for generating ADT messages when an ADT event is triggered.

When a subject is admitted, transferred or discharged in a healthcare facility, the associated millennium domain may send ADT messages through the open interface 106 to a cloud service 108, particularly a clinical data exchange (CDeX) data plane 112 for data governance. The CDeX data plane 112 is a component of the cloud service 108, comprising one or more backend cells 114 and frontend cells 116, for managing flow of data between the healthcare provider domain 102 and the external entities 110. When an ADT message leaves a millennium platform 104a via an open interface 106a, it may enter the backend cell 114, including an ADT inbound 114-1 that may be responsible for receiving and processing ADT messages from external data sources such as the healthcare provider domain 102. For the incoming ADT messages (or ADT feeds), the ADT inbound 114-1 may provide acknowledgment (ACK) indicating the ADT message is successfully received or is valid. If there is an issue (e.g., incorrect data format, missing information), it may send a negative acknowledgment (NACK) message detailing the problem (NACK). Additionally, the backend cell 114 may also include a load balancer 114-2 for distributing ADT messages across different application fleets, thereby facilitating scaling up of the CDeX system to include more healthcare providers and/or to balance incoming traffic.

The backend cell 114 may also be responsible for sharding (or partitioning) the incoming ADT feeds into smaller, more manageable chunks for efficient storage and retrieval.

The ADT feed associated with each healthcare provider may be sharded during on-boarding process. This approach may enable the building of a global patient directory−¿master subject index (MSI) 118-2 that may aggregate medical information and ADT (admission, discharge, transfer) messages associated with the subjects from multiple millennium domains 104 into a unified directory. The engine may a generate a global ID that uniquely identifies a subject across all healthcare provider domain 102. The external entities may use this global ID to search for a subject. The MSI 118-2 may enable a dynamic control and data access as it may allow finding and locating records of a subject across multiple millennium domains 104 for efficient retrieval of data based on request of the external entity 110. It may also serve as the backbone for identifying and maintaining records that which subject's medical data is shared between healthcare provider domain 102 and the external entity 110.

The frontend cell 116 may include components including an engine 118, an ADT outbound 120 and a management API 122. The frontend cell 116 may manage all communication with external entities such as external entities 110. External entities 110 may also be sharded in the frontend cell 116, sharing the same resources, while large external entities may be allocated isolated or dedicated resources. The isolation may restrict the exposing of sensitive data between different external entities while maintaining efficiency. Once the ADT messages are processed in the backend cell 114, it may be passed to the engine 118 included in the frontend cell 116, via an ADT receiver (RX) 118-3. Engine 118 may enable dynamic and selective data sharing by determining dynamically what data should be sent to the external entity 110, based on data governance rules for sharing authorized information. The engine 118 may process the ADT data, retrieve clinical records, and enforce data-sharing permissions based on governance rules.

After determining what information is to be shared, the engine 118 may pass the data to the ADT outbound 120 via an ADT transmit (TX) 116-3 to prepare data for distribution to the external entity 110. The ADT outbound 120 may apply dynamic data sharing to filter out sensitive or restricted data, enabling the sharing of authorized information. The ADT outbound 120 may be responsible for delivering the processed and filtered ADT messages to the ADT inbound endpoints 110-1. The issues incurred in data transmission (e.g., failed deliver) may also be handled by the ADT outbound 120. Once the ADT outbound 120 has completed the task of sending the ADT message, it is delivered to the ADT inbound endpoint 110-1 (e.g., a delivery endpoint of the external entity 110, where it is received by the external entity's system. The ADT inbound endpoint 110-1 may utilize the received clinical information provided by the healthcare provider.

In some aspects, an external entity 110 may interact with the received data through one or more applications 110-2 that may handle tasks such as validating claims, assessing eligibility of the subject or managing care coordination. The payer 102 may be restricted to viewing and processing only the data that is authorized by the data-sharing agreement with the healthcare provider 102, enforced in the CDeX data plane 112. The payer 110 can make requests for additional clinical data such as additional documents related to the encounter such as lab results, imaging reports, etc. or continuity of care documents (CCDs). Using the interoperability interface 118-1 in the engine 118, the payer 110 may submit requests for specific encounters or subject data. The engine 118 may interact with the MSI 118-2 to identify a millennium domain holding the records of the subject. The CDeX data plane 112 may apply data governance rules dynamically to determine what information should be shared based on the permissions. If the requested data is available and authorized by a millennium authorization server 130, the requested data may be fetched from the respective millennium domain via millennium services 132. Subsequently, the engine 118 may generate a response in the form of clinical document architecture (e.g., HL7, HL7 FHIR) or other clinical data formats. The engine 118 may assemble this data and wrap it in a C62 bundle to send it back to the payer 110. Analogous to backend cell 114, the load balancer 114-2 may check that these requests are efficiently handled without overloading any single resource in the backend cell 114 and front cell 116.

When an external entity 110 submits a request to update its resources or alter data access permissions, the CDeX control plane 124 may enable updating the system configuration in real-time. A control plane API 126 may be responsible for maintaining up-to-date configurations of the components of the CDeX system. Upon receiving the request from external entity 110 for updating permissions or access rights, the control plane API 126 may initiate workflows via WaaF (workflow as a function) to update system settings. The CDeX control plane 124 may interact with the management API 122 to push these changes to the frontend cells 116 and backend cells 114 by storing configuration details in a database within the cloud service 108. The backend cells 114 and frontend cells 116, including components such as engine 118, ADT TX 118-4 and the ADT outbound 120, may be dynamically updated with the latest permissions and data-sharing rules. These components may obtain their updated configurations from the management API 122, confirming that any new governance rule, changes in payer agreements, or updated system setting may be immediately applied across the cloud service 108.

For example, if a new payer is onboarded, or if existing payer gains new access rights, the CDeX control plane 124 may verify that these changes are dynamically reflected in the system. Similarly, if data-sharing permissions are revoked, the system may automatically apply these changes, preventing unauthorized access to the clinical data. The frontend cell may be responsible for handling requests from the payer, where multiple external entities may share the frontend cell. Alternatively, for large external entities dedicated resources may be allocated for isolation and better performance. The control plane may include one or more APIs (application programming interfaces).

Apart from facilitating and providing a CDeX data plane and control plane, the cloud service 108 may also provide cloud support services 134 to support and facilitate the working of CDeX system. For example, the cloud support services 134 may include KaaS (Kiev-as-a-service) 134-1 for storing CDeX control plane metadata and IAM 134-3 (identity and access management) services to manage user identities, control access to resources, and enforce security policies within an organization. Additionally, WFaaS 134-4 for orchestrating customers and clients workflows triggered by APIs, a vault 134-2 or key management services (KMS) for storing service secrets i.e., database credentials.

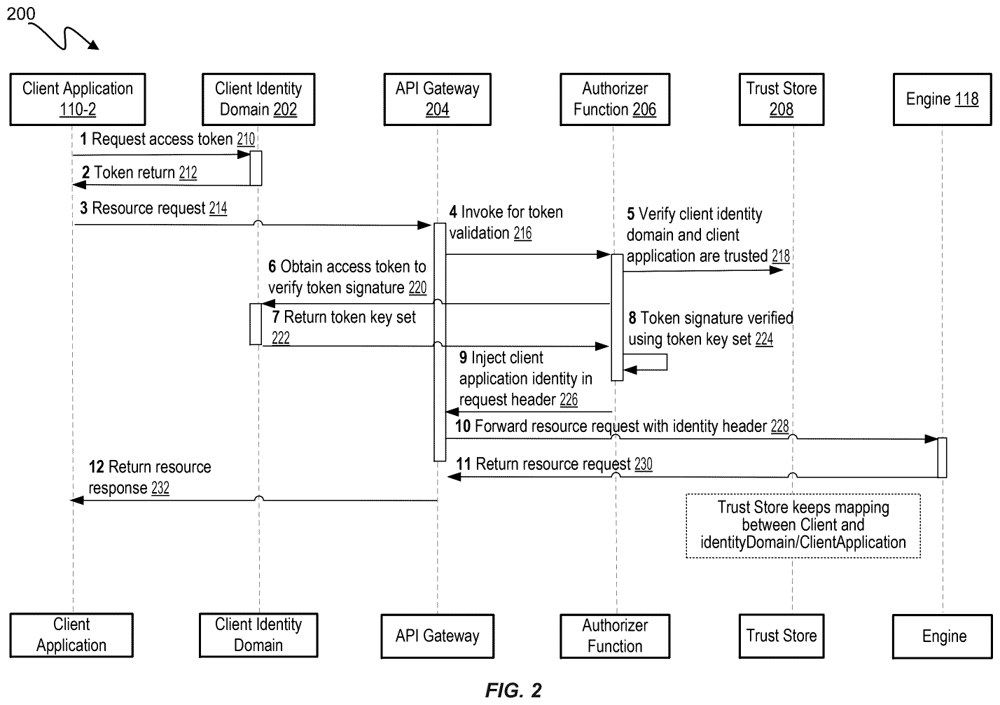

FIG. 2 illustrates an exemplary sequence diagram authenticating a client application in accordance with some aspects of the present disclosure. In the disclosed healthcare interoperability framework, a payer user, such as a developer, system administrator or a staff member, may initiate a process by creating a confidential application within one of the designated client identity domains in the payer tenancy within the cloud service 108. This application is termed “confidential” as it is designed to securely handle sensitive data and perform strict authentication and authorization measures to protect subject information. The identity domain may refer to a logical grouping of user identities and associated security policies, allowing organizations to manage access controls effectively through identity provider−¿a service responsible for authenticating users and applications, issuing tokens that grant access to specific resources. In this framework, various authentication protocols may be used e.g., OAuth 2.0 protocol is utilized to securely manage access tokens, enabling applications to obtain limited access to user data without exposing sensitive credentials. Other standards, such as OpenID Connect for identity verification and HL7 FHIR for data exchange, may also be employed to enhance interoperability and security within healthcare systems.

In FIG. 2, the exemplary sequence diagram detailing the interaction between a client application 110-2, a client identity domain 202, an application programming interface (API) gateway 204, an authorizer function 206, a trust store 208, and the engine 118 within a cloud-based centralized clinical data exchange system (CDeX). In this embodiment, the sequence begins with the client application 110-2 initiating a request 214 for an access token from the client identity domain 202, using the authentication protocol. Once the token is granted at 212, the client application may send a resource request, including the access token, at 214. The API gateway 204 may then invoke the authorizer function 206 for token validation and decoding of the access token e.g., in a JWT (JSON web token) format, at 216. The authorizer function 206 may verify the client identity domain 202 and the trustworthiness of the client application, at 218, to check whether both are trusted entities with allowed scopes. Upon verification, at 220, the authorizer function may obtain the access token and verify the token signature with a collection of keys or a key set. For example, if the access token is JWT then the authorizer function 206 may perform verification using a JWK (JSON web key) set that is returned from the trust store, at 230. The trust store 208 may maintain mappings between the client identity domain 202 and client application 110-2.

Once the token signature is validated, the client application identity (i.e., application identifier (ID) and/or permissions) may be injected into a request header resulting in an identity header, at 226, by the authorizer function 206. This identity header along with the resource request may be forwarded, at 228, to the engine 118 for processing. The engine 118 may process the resource request and send the response, at 230, via the API gateway 204 back to the client application 110-2, completing the sequence at 232. The response may include the requested resource from the specific healthcare provider after being processed and validated by the engine 118 in accordance with the data governance rules and permissions granted to the payer. The trust store 208 may act as an intermediary, enabling trusted mappings between client application 110-2 and the client identity domains 202. The authorizer function 206 may operate in a serverless environment within the cloud service 108 and integrated with the API gateway 204 to dynamically enforce authentication and authorization policies based on token validation. This mechanism may facilitate a secure and efficient exchange of clinical data between healthcare provider domain 102 and external entities 110, allowing for seamless access to the subject clinical data, while maintaining strict access control in compliance with the authorization protocols e.g., OAuth2.0 protocol and FHIR standards.

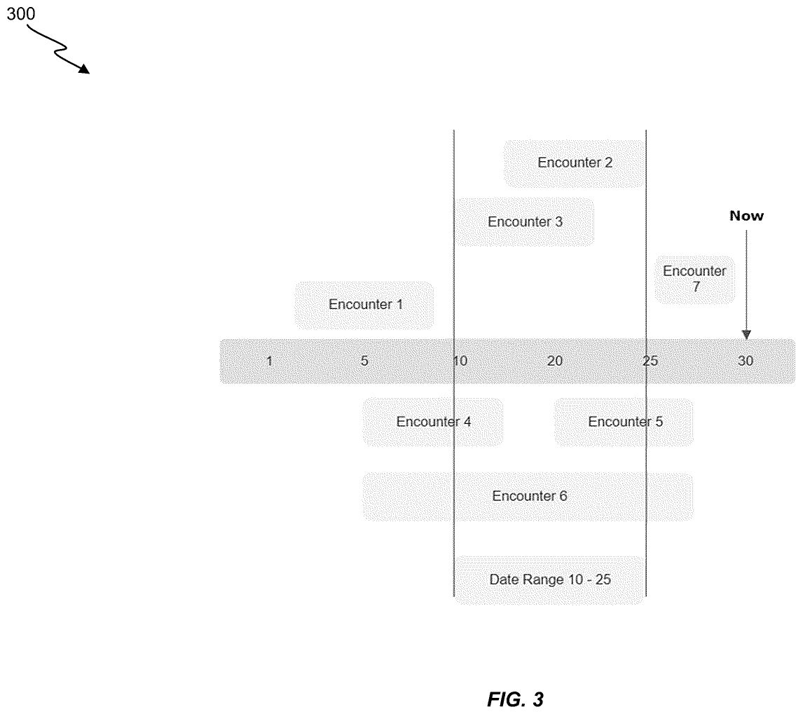

FIG. 3 illustrates a graphical representation of selective data access depicting subject encounters over a specified date range (e.g., spanning a 30-day window), where individual encounters are shown as horizontally shaded blocks representing periods of subject interaction with a healthcare provider. In this example, the encounters may correspond to events such as admissions, discharges, or other healthcare interactions, each governed by specific ADT message types. For example, the encounters may be denoted as “Encounter 1”, “Encounter 2”, . . . , “Encounter 7”. In FIG. 3, a query with a date range of 10 to 25 is used to define which subject encounters fall within this window. The query may be initiated by a payer, constrained by a 30-day window limit, designed to provide a manageable and focused subset of subject data for the purpose of analysis, reviewing, reporting, or other operational processes. The encounters that partially or fully overlap with this defined date range may be included in the query results. Specifically, “Encounters 2, 3, 4, 5”, and “Encounter 6” are included because they have some temporal overlap with the defined date range, as indicated by the horizontal positioning in the graphical representation 300. Encounters that do not overlap this range, such as “Encounter 1” and “Encounter 7”, may be excluded from the query results. These encounters may fall outside the specified time window enabling that only relevant data is processed.

This dynamic windowed selection of encounter times may improve the handling and retrieval of healthcare data, specifically in systems that handle large volumes of subject information. By limiting the query to a specific time frame, in this example a 30-day window, the system can efficiently narrow down the dataset, making it feasible to extract meaningful insights without being overwhelmed by irrelevant or outdated information. This process may be particularly relevant for healthcare provider domain 102 and external entities 110 (e.g., insurers) that may need to access dynamic patient data in real time, as it may enable that only the most pertinent information—such as ongoing treatments, active admissions, and recent discharges—is retrieved and shared according to the permissions defined by the system. Additionally, including open encounters (i.e., encounters that have not yet been concluded) may enable the system capable of monitoring ongoing care and making informed decisions about a subject's current health status and care needs.

This approach may be significant where real-time data access is required to determine the course of care, billing cycles, or compliance with regulatory frameworks. For instance, external entities may need to access subject encounters that are relevant to a particular billing period, while healthcare providers may require up-to-date information on ongoing patient care to avoid lapses in treatment or medication administration. The defined window-based query may enable such selective data access, aligning with both clinical and operational objectives in healthcare environments.

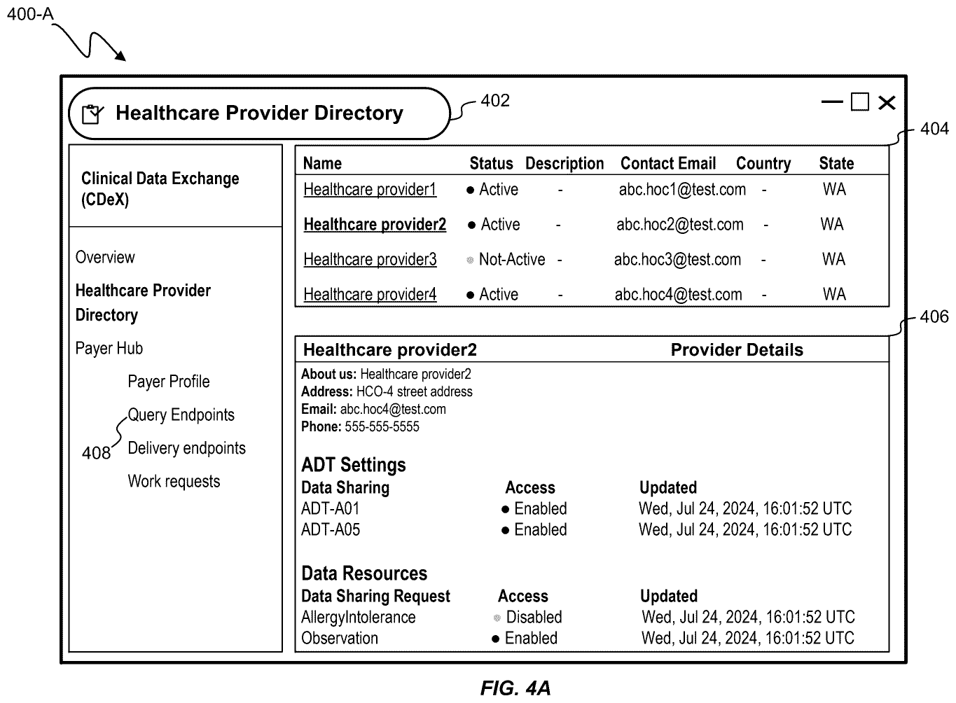

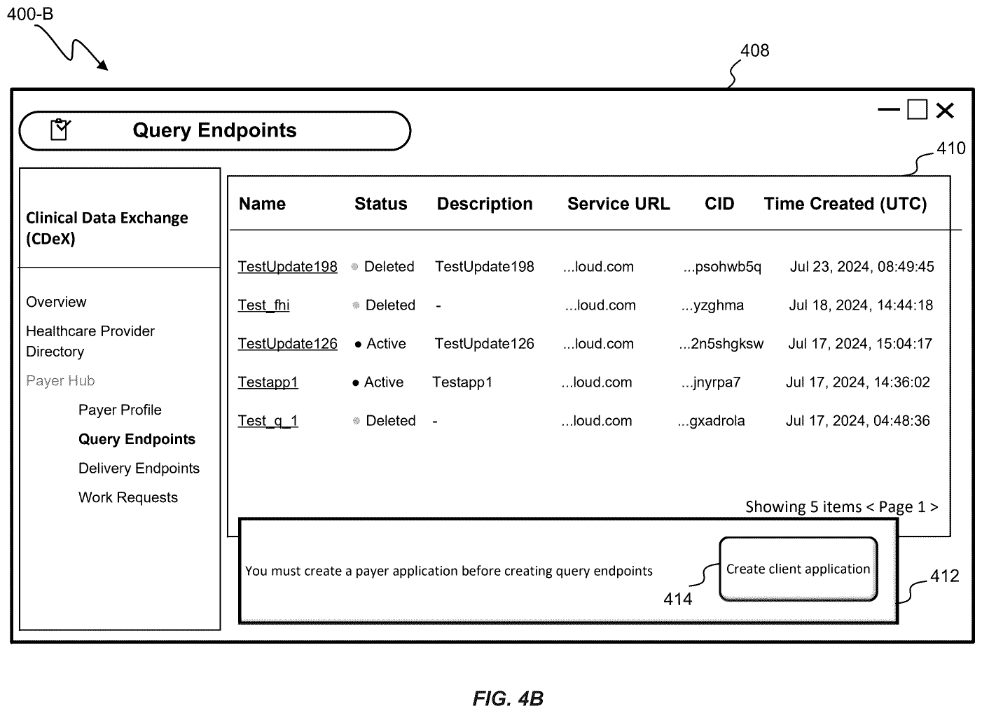

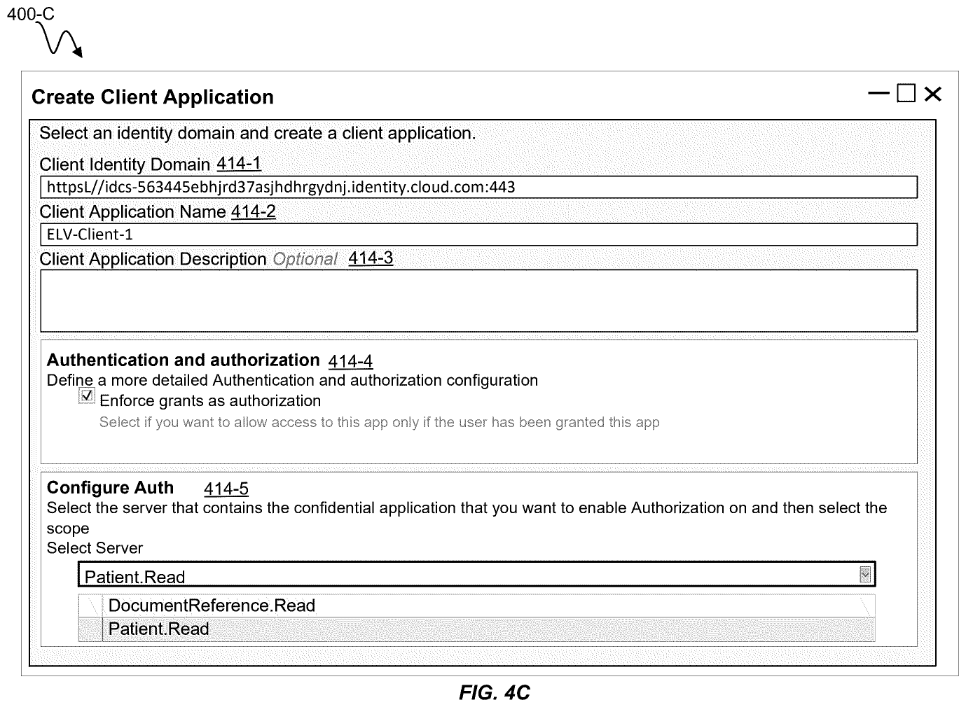

FIG. 4A illustrates an exemplary graphical user interface (GUI) of an external entity 400, showing a healthcare provider directory 402. The techniques, as disclosed herein, may include clinical data exchange (CDeX) representing a multi-tenant cloud-based service comprising the CDeX data plane 112, CDeX control plane 124 and additional components such as cloud console. For registering with the CDeX, the external entities may require a cloud tenancy, where the external entities 110 may be registered after completing authorization, providing proof belonging to the claimed payer e.g., health insurance company. The exchange of clinical data and ADT messages between the healthcare provider domains and payer domains may be enabled via a unified interoperability interface 118-1 within the CDeX. Once an external entity e.g., a payer has been registered with the CDeX, it may access (on-boarded) healthcare providers, via the exemplary GUI 400, in the table 404 along with additional information such as contact information or status as being active or inactive. By selecting a healthcare provider from the table 404, it may further display details associated with the healthcare provider such as assigned cloud identifiers, ADT settings as to what messages or encounter information to share. For example, “ADT-01” in the table 406, may represent ADT messages associated with admit/visit encounter.

Additionally, the healthcare provider may also define data sharing configurations or data governance rules for each external entity separately. These settings may be seen by the external entity, the, via the exemplary GUI 400, in the healthcare providers directory. For example, the table 406 displays requested data resources (or clinical data to share) from the selected healthcare provider and the corresponding access status, for example, “AllergyIntolerance” may represent clinical data associated with allergies and “Observation” may represent request for vital signs, lab results, device measurements (i.e., electrocardiogram or EKG). Similarly, other data resources may include clinical notes, goals, care plan, inpatient and outpatient medications orders or requests, and other clinical data.

In some aspects, a unified interoperability interface may be provided to enable seamless exchange of clinical data across multiple EHR systems associated with the healthcare providers domains, regardless of the underlying infrastructure. The process may begin with the creation of a client application within the client identity domain, where both the client identity domain and client application ID are provided during the creation of a query endpoint. The client identity domain may refer to a domain assigned to an external entity within the cloud service 108. Secure authentication may be achieved through an authentication protocol, where the client application may obtain an access token from the identity provider using credentials of client application. This token may be verified to authenticate the external entity and confirm access rights before any data request can be processed. Once the authentication is complete, the external entity can submit requests through the query endpoint. The engine 118, which manages the processing of resource requests, may expose resources such as “/Subject” for subject identification based on demographic information, and “/DocumentReference” for retrieving clinical documents, including standardized formats such as the consolidated clinical document architecture (C-CDA). These resources may enable the retrieval of subject data, enabling consistency in document formatting and content for clinical encounters.

The external entity may be allowed to make multiple concurrent requests, with limits enforced at the API Gateway level to manage the volume of queries per resource type and per query endpoint. In examples, where requests cannot be fulfilled within a set timeout period (e.g., 120 seconds), the engine 118 may temporarily cache the results retrieved from the healthcare providers, allowing the external entity to retry the request and benefit from the cached data, reducing latency and improving efficiency. To access relevant and requested clinical data, the engine 118 may identify healthcare providers that hold subject medical records based on the MSI. The records may be filtered using subject-specific and/or participant-specific data permissions and/or data governance rules, so that only permitted and accurate data is shared according to the external entity's authorization. The engine 118 may locate the encounters associated with the subject and verify the association with the external entity using coverage information stored in an encounter coverage resource, which may link to the entity's insurance organization ID. Once the relevant encounters are identified, clinical data may be fetched, via the engine 118, through secure calls to services, such as the millennium HL7 FHIR services, which process the request based on the applicable data governance policies.

The retrieved clinical data may be bundled and returned in a consistent, standardized format. This may include HL7 CDA R2.1 documents for each encounter and additional documents, such as unstructured C62 records, so that the external entity receives complete and actionable information. These documents may be generated and validated against industry standards to maintain uniformity in the data exchanged between external entities and healthcare providers, thereby providing requested resource information such as allergies, medications, and other clinical details that are captured consistently across different health domains. In other examples where existing systems, such as millennium or EHRs, may generate incomplete or inconsistent records due to varying configurations or versions, the interoperability interface 118-1 may enable normalization of these documents before exchange. By implementing a standardized approach to document creation and delivery—such as enabling proper formatting and population of C-CDA documents—the CDeX system may provide all external entities, high-quality clinical data that is both accurate and requested, regardless of the underlying source.

FIG. 4B illustrates an exemplary graphical user interface 400-B (interchangeably referred to as 408) showing a list of query endpoints in the table 410, from the FIG. 4A. The query endpoints may allow an external entity to set up an interoperability endpoint within the centralized clinical data exchange (CDeX) for retrieving clinical data from a specific healthcare provider. The clinical data may be in the form of standardized clinical documents such as C-CDA. The table 410 shows information associated with one or more query endpoints, such as, query endpoint names, status, a service uniform resource locator (URL) that refers to the interoperability endpoint, a cloud identifier (ID) and the time of creation of an endpoint. Before creating a query endpoint, the external entity (such as payer) creates a client application in one of the client identity domains in which scope and associated configurations are defined. A notification 412 may appear, if the client application is not created before creation of query endpoints.

FIG. 4C shows an exemplary GUI 400-C, providing a console for creating a client application for an external entity (e.g., a payer). The console may input a client identity domain 414-1, a client application name 414-2, an optional description of client application 414-3, a section enabling authentication and authorization of the client application 414-4, and selecting a server 414-5. Selection of a server may represent the specific environment or instance where the client application will be hosted or run. Alternatively, it may represent selecting a tenancy associated with an external entity, which is a dedicated space within a multi-tenant architecture that represents, in this example, the payer tenancy holding the client application. The client application may be authorized and authenticated by the process, as described in reference to the FIG. 2.

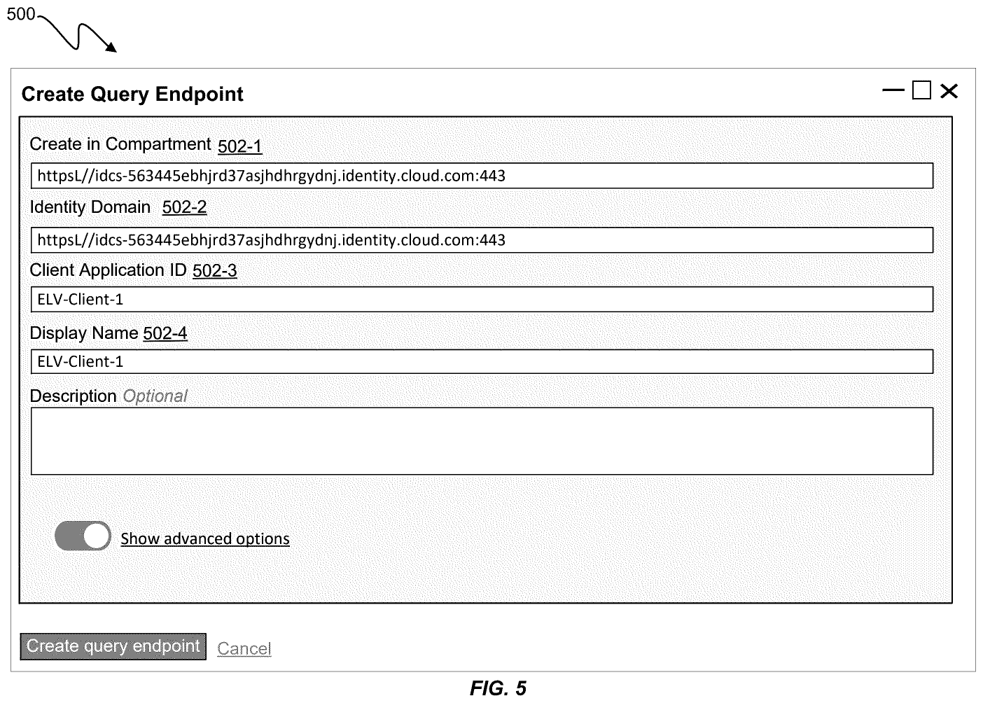

After the creation of the client application, the query endpoint may be created. FIG. 5 illustrates an exemplary GUI, providing a console 500 for creating the query endpoint to the external entity. The console 500 displays different input parameters to be taken, for example, the external entity may be asked to input a compartment 502-1, a client identity domain 502-2 e.g., by providing a URL, client application ID 502-3, query endpoint name 502-4, and an optional description. Specifying compartment may represent a logical container to which the endpoints may be associated, defining the boundaries within which that endpoint will operate. This may include considerations for access control (e.g., granted permissions to interact with query endpoints based on their roles), billing, and resource management.

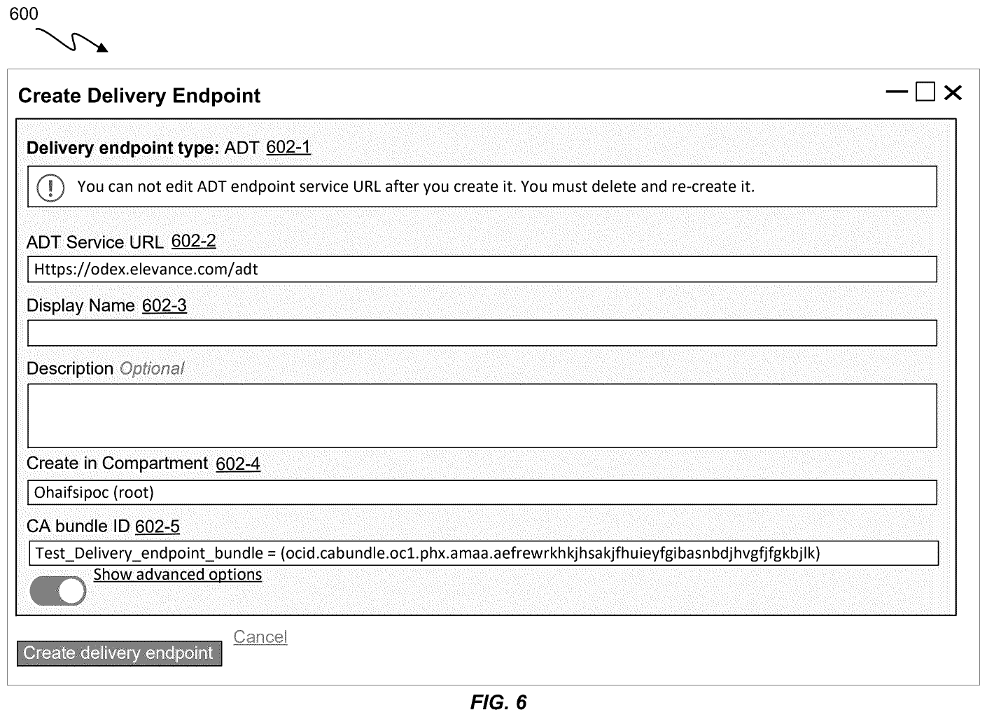

FIG. 6 illustrates an exemplary GUI, providing a console 600 for creating a delivery endpoint for the external entity. This delivery endpoint may be configured within the ADT inbound endpoint 110-1 to receive the requested resource according to the data governance rules and data-sharing permissions set by the healthcare provider, in response to the requests made through the query endpoints. Through delivery endpoint, the external entity specifies an endpoint where CDeX system can push ADT feed (e.g., HL7 messages) from the healthcare provider. Similar to the creation of query endpoint, a delivery endpoint may be created through the console 600 by providing input parameters, such as endpoint type 602-1 (as ADT), a service URL 602-2, name of delivery endpoint 602-3, compartment 602-4 and CA (certificate authority) bundle ID 602-5.

The API for creating the delivery endpoint may require the external entities to provide a digital certificate identifier (i.e., CA bundle ID referring to a collection of trusted certificates from various certification authorities) for validating server certificates when initiating secure sessions, such as mTLS. The external entities may be responsible for managing and updating their certificates as necessary, whereas the ADT outbound 120 within CDeX data plane 112 may be responsible for validating the server certificates. It may be recommended that external entities verify the common name associated with the certificates for added security. After creation of endpoints, a communication channel may be established by connecting query endpoints with the delivery endpoint that can facilitate real-time ADT feed and clinical data exchange associated with a specific subject between the designated external entity and healthcare provider.

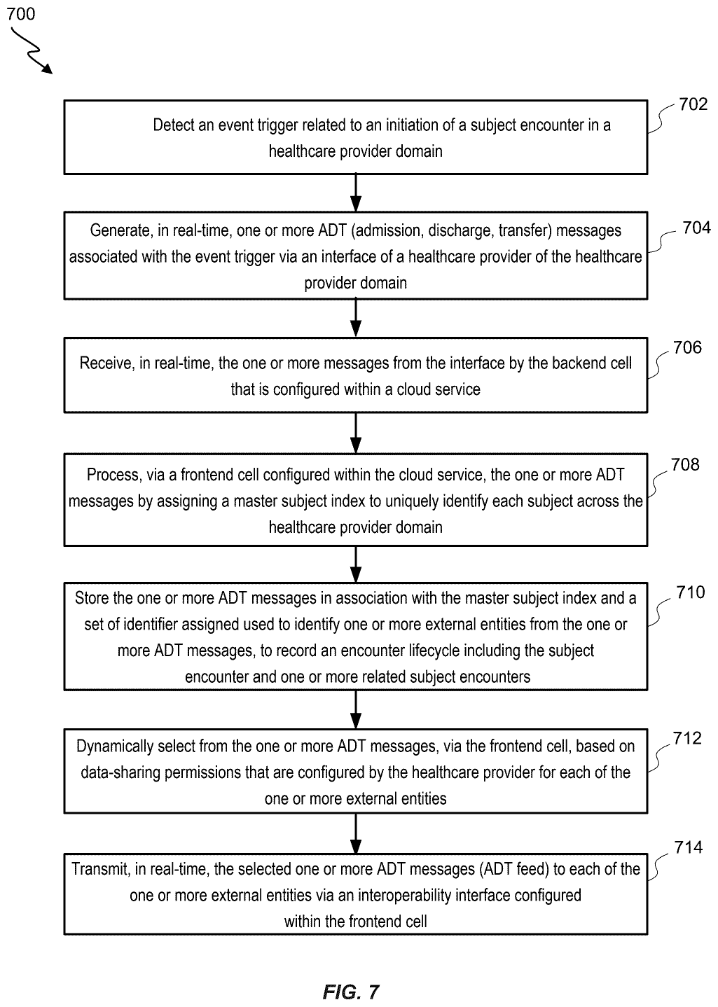

FIG. 7 illustrates an exemplary workflow 700 of notifying participants or external entities (e.g., regulators, payers, insurance companies or other entities) in real-time or near real-time when a subject encounter occurs. The participants may subscribe to clinical, or ADT events updates based on subject-specific dynamic and selective data access between a healthcare provider domain and each individual participant. The blocks in the exemplary workflow 700 are illustrated in a specific order, while the order can be modified, for example, some blocks may be performed before others, and some blocks may be performed simultaneously. The blocks can be performed by hardware, software, or a combination thereof. The process may begin, at block 702, by detecting an event trigger that indicates the initiation of a subject encounter within a healthcare provider domain 102. In real-time, one or more ADT messages may be generated, at block 704, in response to the event trigger through an interface (e.g., open interface 106) of the healthcare provider, which is designed to transmit the ADT messages to a backend cell 114.

The backend cell 114, configured within a cloud service 108, may receive these ADT messages in real-time, at block 706. Subsequently, a frontend cell 116, also within the cloud service 108, may process the ADT messages by assigning a master subject index that uniquely identifies each subject across the healthcare provider domain, at block 708. The frontend cell 116 may identify one or more external entities from the ADT messages based on identifiers assigned during processing. The ADT messages may then be stored alongside the master subject index and identifiers to document the encounter lifecycle, which includes the subject encounter and any related encounters, at block 710. Furthermore, the frontend cell 116 may dynamically select ADT messages based on subject-based data-sharing permissions configured by the healthcare provider for each external entity, at block 712. Finally, the selected ADT messages may be transmitted in real-time to the identified external entities 110 through an interoperability interface 118-1 configured within the frontend cell 116, at block 714.

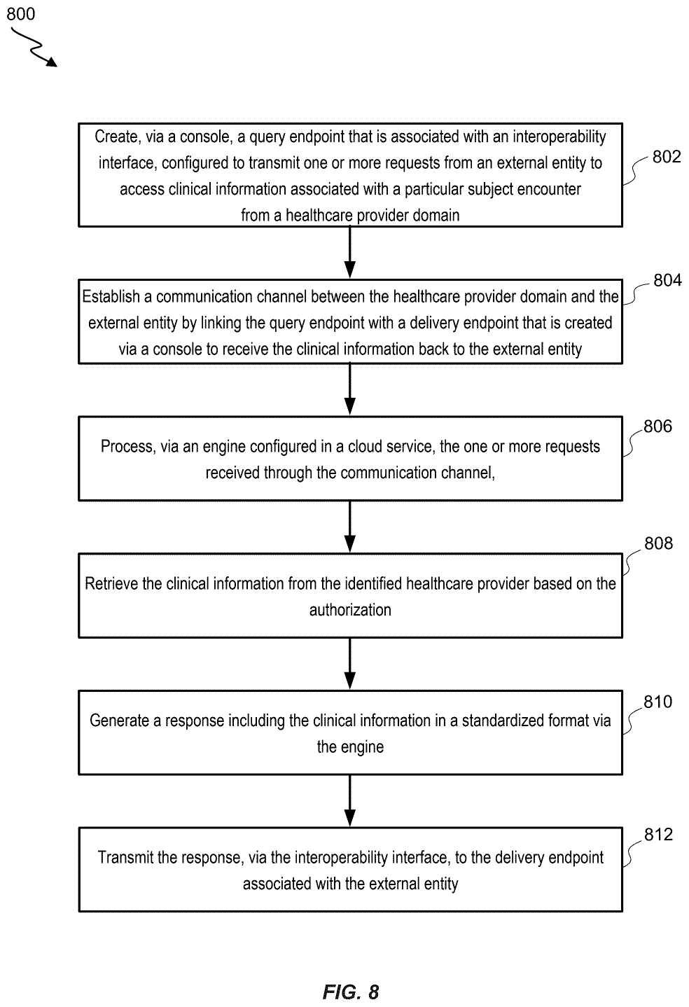

FIG. 8 illustrates an exemplary workflow 800 of a unified interoperability interface 118-1 configured in a cloud service 108 to operate seamlessly across multiple millennium platforms 104a, . . . , 104n or EHR systems, enabling the exchange of clinical data regardless of the underlying infrastructure. The blocks in the exemplary workflow 800 are illustrated in a specific order, while the order can be modified, for example, some blocks may be performed before others, and some blocks may be performed simultaneously. The blocks can be performed by hardware, software, or a combination thereof. The process may begin, at block 802, by creating a query endpoint associated with an interoperability interface 118-1. The query endpoint may be configured to send requests from an external entity for accessing clinical information related to a specific subject encounter within a healthcare provider domain 102.

A delivery endpoint may also be created via the console to facilitate receiving the clinical information back to the external entity. This setup may establish a communication channel between the healthcare provider domain 102 and the external entity by connecting the query and delivery endpoints for a subject involved in the particular subject encounter, at block 804. An engine 118, configured in the cloud service 108, may process the requests received through this communication channel, at block 806, by identifying the subject and the corresponding healthcare provider holding the clinical information using a master subject index (MSI) 118-2 that uniquely identifies the subject across the healthcare provider domain 102. The engine 118 then may apply subject-specific data-sharing permissions to verify whether the clinical information can be shared with the external entity. Upon authorization, the engine 118 may retrieve the verified clinical information from the identified healthcare provider, at block 808. At block 810, a response may be generated that includes the verified clinical information in a standardized format, and may transmit the response to the delivery endpoint associated with the external entity via the interoperability interface 118-1, at block 812.

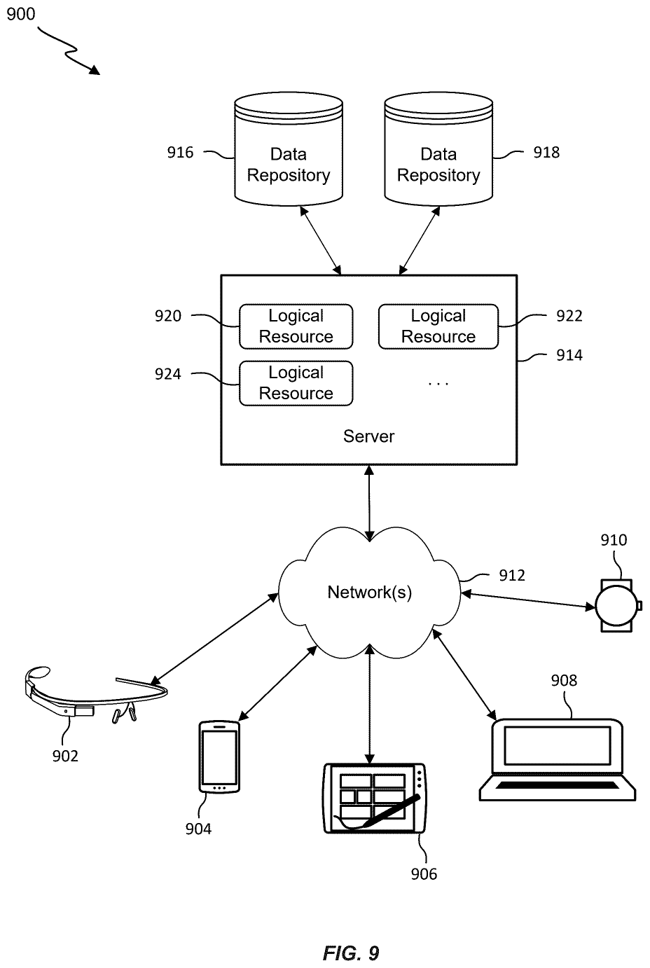

FIG. 9 depicts a simplified diagram of a distributed system 900 for implementing an embodiment. In the illustrated embodiment, distributed system 900 includes one or more client computing devices 902, 904, 906, 908, and/or 910 coupled to a server 914 via one or more communication networks 912. Clients computing devices 902, 904, 906, 908, and/or 910 may be configured to execute one or more applications. In various aspects, server 914 may be adapted to run one or more services or software applications that enable techniques for the centralized clinical data exchange (CDeX). In certain aspects, server 914 may also provide other services or software applications that can include non-virtual and virtual environments. In some aspects, these services may be offered as web-based or cloud services, such as under a Software as a Service (SaaS) model to the users of client computing devices 902, 904, 906, 908, and/or 910. Users operating client computing devices 902, 904, 906, 908, and/or 910 may in turn utilize one or more client applications to interact with server 914 to utilize the services provided by these components.

In the configuration depicted in FIG. 9, server 914 may include one or more components 920, 922 and 924 that implement the functions performed by server 914. These components may include software components that may be executed by one or more processors, hardware components, or combinations thereof. It should be appreciated that various different system configurations are possible, which may be different from distributed system 900. The embodiment shown in FIG. 9 is thus one example of a distributed system for implementing an embodiment system and is not intended to be limiting.

Users may use client computing devices 902, 904, 906, 908, and/or 910 for techniques in accordance with the teachings of this disclosure. A client device may provide an interface that enables a user of the client device to interact with the client device. The client device may also output information to the user via this interface. Although FIG. 9 depicts only five client computing devices, any number of client computing devices may be supported.

The client devices may include various types of computing systems such as smart phones or other portable handheld devices, general purpose computers such as personal computers and laptops, workstation computers, personal assistant devices, smart watches, smart glasses, or other wearable devices, equipment firmware, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux® or Linux-like operating systems such as Oracle® Linux and Google Chrome® OS) including various mobile operating systems (e.g., Microsoft Windows Mobile®, iOS®, Windows Phone®, Android®, HarmonyOS®, Tizen®, KaiOS®, Sailfish® OS, Ubuntu® Touch, CalyxOS®). Portable handheld devices may include cellular phones, smartphones, (e.g., an iPhone®), tablets (e.g., iPad®), and the like. Virtual personal assistants such as Amazon® Alexa®, Google® Assistant, Microsoft® Cortana®, Apple® Siri®, and others may be implemented on devices with a microphone and/or camera to receive user or environmental inputs, as well as a speaker and/or display to respond to the inputs.

Wearable devices may include Apple® Watch, Samsung Galaxy® Watch, Meta Quest®, Ray-Ban® Meta® smart glasses, Snap® Spectacles, and other devices. Gaming systems may include various handheld gaming devices, Internet-enabled gaming devices (e.g., a Microsoft Xbox® gaming console with or without a Kinect® gesture input device, Sony PlayStation® system, Nintendo Switch®, and other devices), and the like. The client devices may be capable of executing various different applications such as various Internet-related apps, communication applications (e.g., e-mail applications, short message service (SMS) applications) and may use various communication protocols.

Network(s) 912 may be any type of network familiar to those skilled in the art that can support data communications using any of a variety of available protocols, including without limitation TCP/IP (transmission control protocol/Internet protocol), SNA (systems network architecture), IPX (Internet packet exchange), AppleTalk®, and the like. Merely by way of example, network(s) 912 can be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Server 914 may be composed of one or more general purpose computers, specialized server computers (including, by way of example, PC (personal computer) servers, UNIX® servers, LINIX® servers, mid-range servers, mainframe computers, rack-mounted servers, etc.), server farms, server clusters, a Real Application Cluster (RAC), database servers, or any other appropriate arrangement and/or combination. Server 914 can include one or more virtual machines running virtual operating systems, or other computing architectures involving virtualization such as one or more flexible pools of logical storage devices that can be virtualized to maintain virtual storage devices for the server. In various aspects, server 914 may be adapted to run one or more services or software applications that provide the functionality described in the foregoing disclosure.

The computing systems in server 914 may run one or more operating systems including any of those discussed above, as well as any commercially available server operating system. Server 914 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, SAP®, Amazon®, Sybase®, IBM® (International Business Machines), and the like.

In some implementations, server 914 may include one or more applications to analyze and consolidate data feeds and/or event updates received from users of client computing devices 902, 904, 906, 908, and/or 910. As an example, data feeds and/or event updates may include, but are not limited to, blog feeds, Threads® feeds, Twitter® feeds, Facebook® updates or real-time updates received from one or more third party information sources and continuous data streams, which may include real-time events related to sensor data applications, financial tickers, network performance measuring tools (e.g., network monitoring and traffic management applications), clickstream analysis tools, automobile traffic monitoring, and the like. Server 914 may also include one or more applications to display the data feeds and/or real-time events via one or more display devices of client computing devices 902, 904, 906, 908, and/or 910.

Distributed system 900 may also include one or more data repositories 916, 918. These data repositories may be used to store data and other information in certain aspects. For example, one or more of the data repositories 916, 918 may be used to store information for techniques disclosed herein. Data repositories 916, 918 may reside in a variety of locations. For example, a data repository used by server 914 may be local to server 914 or may be remote from server 914 and in communication with server 914 via a network-based or dedicated connection. Data repositories 916, 918 may be of different types. In certain aspects, a data repository used by server 914 may be a database, for example, a relational database, a container database, an Exadata® storage device, or other data storage and retrieval tools such as databases provided by Oracle Corporation® and other vendors. One or more of these databases may be adapted to enable storage, update, and retrieval of data to and from the database in response to structured query language (SQL)-formatted commands.

In certain aspects, one or more of data repositories 916, 918 may also be used by applications to store application data. The data repositories used by applications may be of different types such as, for example, a key-value store repository, an object store repository, or a general storage repository supported by a file system.

In one embodiment, server 914 is part of a cloud-based system environment in which various services may be offered as cloud services 108, for a single tenant or for multiple tenants where data, requests, and other information specific to the tenant are kept private from each tenant. In the cloud-based system environment, multiple servers may communicate with each other to perform the work requested by client devices from the same or multiple tenants. The servers communicate on a cloud-side network that is not accessible to the client devices in order to perform the requested services and keep tenant data confidential from other tenants.

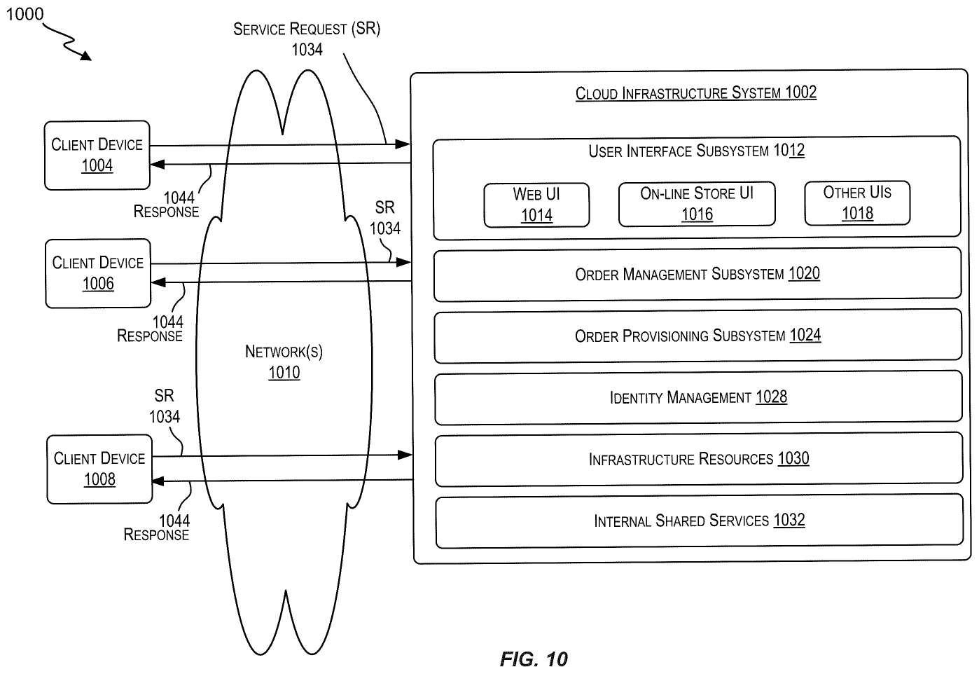

FIG. 10 is a simplified block diagram of a cloud-based system environment in accordance with certain aspects of various embodiments of the invention. In the embodiment depicted in FIG. 10, cloud infrastructure system 1002 may provide one or more cloud services that may be requested by users using one or more client computing devices 1004, 1006, and 1008. Cloud infrastructure system 1002 may comprise one or more computers and/or servers that may include those described above for server 914. The computers in cloud infrastructure system 1002 may be organized as general purpose computers, specialized server computers, server farms, server clusters, or any other appropriate arrangement and/or combination.

Network(s) 1010 may facilitate communication and exchange of data between clients 1004, 1006, and 1008 and cloud infrastructure system 1002. Network(s) 1010 may include one or more networks. The networks may be of the same or different types. Network(s) 1010 may support one or more communication protocols, including wired and/or wireless protocols, for facilitating the communications.

The embodiment depicted in FIG. 10 is only one example of a cloud infrastructure system and is not intended to be limiting. It should be appreciated that, in some other aspects, cloud infrastructure system 1002 may have more or fewer components than those depicted in FIG. 10, may combine two or more components, or may have a different configuration or arrangement of components. For example, although FIG. 10 depicts three client computing devices, any number of client computing devices may be supported in alternative aspects.

The term cloud service is generally used to refer to a service that is made available to users on demand and via a communication network such as the Internet by systems (e.g., cloud infrastructure system 1002) of a service provider. Typically, in a public cloud environment, servers and systems that make up the cloud service provider's system are different from the cloud customer's (“tenant's”) own on-premises servers and systems. The cloud service provider's systems are managed by the cloud service provider. Tenants can thus avail themselves of cloud services provided by a cloud service provider without having to purchase separate licenses, support, or hardware and software resources for the services. For example, a cloud service provider's system may host an application, and a user may, via a network 1010 (e.g., the Internet), on demand, order and use the application without the user having to buy infrastructure resources for executing the application. Cloud services are designed to provide easy, scalable access to applications, resources, and services. Several providers offer cloud services. For example, several cloud services are offered by Oracle Corporation®, such as database services, middleware services, application services, and others.

In certain aspects, cloud infrastructure system 1002 may provide one or more cloud services using different models such as under a Software as a Service (SaaS) model, a Platform as a Service (PaaS) model, an Infrastructure as a Service (IaaS) model, a Data as a Service (DaaS) model, and others, including hybrid service models. Cloud infrastructure system 1002 may include a suite of databases, middleware, applications, and/or other resources that enable provision of the various cloud services.

A SaaS model enables an application or software to be delivered to a tenant's client device over a communication network like the Internet, as a service, without the tenant having to buy the hardware or software for the underlying application. For example, a SaaS model may be used to provide tenants access to on-demand applications that are hosted by cloud infrastructure system 1002. Examples of SaaS services provided by Oracle Corporation® include, without limitation, various services for human resources/capital management, client relationship management (CRM), enterprise resource planning (ERP), supply chain management (SCM), enterprise performance management (EPM), analytics services, social applications, and others.

An IaaS model is generally used to provide infrastructure resources (e.g., servers, storage, hardware, and networking resources) to a tenant as a cloud service to provide elastic compute and storage capabilities. Various IaaS services are provided by Oracle Corporation®.

A PaaS model is generally used to provide, as a service, platform and environment resources that enable tenants to develop, run, and manage applications and services without the tenant having to procure, build, or maintain such resources. Examples of PaaS services provided by Oracle Corporation® include, without limitation, Oracle Database Cloud Service (DBCS), Oracle Java Cloud Service (JCS), data management cloud service, various application development solutions services, and others.

A DaaS model is generally used to provide data as a service. Datasets may searched, combined, summarized, and downloaded or placed into use between applications. For example, user profile data may be updated by one application and provided to another application. As another example, summaries of user profile information generated based on a dataset may be used to enrich another dataset.

Cloud services are generally provided on an on-demand self-service basis, subscription-based, elastically scalable, reliable, highly available, and secure manner. For example, a tenant, via a subscription order, may order one or more services provided by cloud infrastructure system 1002. Cloud infrastructure system 1002 then performs processing to provide the services requested in the tenant's subscription order. Cloud infrastructure system 1002 may be configured to provide one or even multiple cloud services.

Cloud infrastructure system 1002 may provide the cloud services via different deployment models. In a public cloud model, cloud infrastructure system 1002 may be owned by a third party cloud services provider and the cloud services are offered to any general public tenant, where the tenant can be an individual or an enterprise. In certain other aspects, under a private cloud model, cloud infrastructure system 1002 may be operated within an organization (e.g., within an enterprise organization) and services provided to clients that are within the organization. For example, the clients may be various departments or employees or other individuals of departments of an enterprise such as the Human Resources department, the Payroll department, etc., or other individuals of the enterprise. In certain other aspects, under a community cloud model, the cloud infrastructure system 1002 and the services provided may be shared by several organizations in a related community. Various other models such as hybrids of the above mentioned models may also be used.

Client computing devices 1004, 1006, and 1008 may be of different types (such as devices 902, 904, 906, and 908 depicted in FIG. 9) and may be capable of operating one or more client applications. A user may use a client device to interact with cloud infrastructure system 1002, such as to request a service provided by cloud infrastructure system 1002.

In some aspects, the processing performed by cloud infrastructure system 1002 for providing chatbot services may involve big data analysis. This analysis may involve using, analyzing, and manipulating large data sets to detect and visualize various trends, behaviors, relationships, etc. within the data. This analysis may be performed by one or more processors, possibly processing the data in parallel, performing simulations using the data, and the like. For example, big data analysis may be performed by cloud infrastructure system 1002 for determining the intent of an utterance. The data used for this analysis may include structured data (e.g., data stored in a database or structured according to a structured model) and/or unstructured data (e.g., data blobs (binary large objects)).

As depicted in the embodiment in FIG. 10, cloud infrastructure system 1002 may include infrastructure resources 1030 that are utilized for facilitating the provision of various cloud services offered by cloud infrastructure system 1002. Infrastructure resources 1030 may include, for example, processing resources, storage or memory resources, networking resources, and the like.

In certain aspects, to facilitate efficient provisioning of these resources for supporting the various cloud services provided by cloud infrastructure system 1002 for different tenants, the resources may be bundled into sets of resources or resource modules (also referred to as “pods”). Each resource module or pod may comprise a pre-integrated and optimized combination of resources of one or more types. In certain aspects, different pods may be pre-provisioned for different types of cloud services. For example, a first set of pods may be provisioned for a database service, a second set of pods, which may include a different combination of resources than a pod in the first set of pods, may be provisioned for Java service, and the like. For some services, the resources allocated for provisioning the services may be shared between the services.

Cloud infrastructure system 1002 may itself internally use services 1032 that are shared by different components of cloud infrastructure system 1002 and which facilitate the provisioning of services by cloud infrastructure system 1002. These internal shared services may include, without limitation, a security and identity service, an integration service, an enterprise repository service, an enterprise manager service, a virus scanning and whitelist service, a high availability, backup and recovery service, service for enabling cloud support, an email service, a notification service, a file transfer service, and the like.

Cloud infrastructure system 1002 may comprise multiple subsystems. These subsystems may be implemented in software, or hardware, or combinations thereof. As depicted in FIG. 10, the subsystems may include a user interface subsystem 1012 that enables users of cloud infrastructure system 1002 to interact with cloud infrastructure system 1002. User interface subsystem 1012 may include various different interfaces such as a web interface 1014, an online store interface 1016 where cloud services provided by cloud infrastructure system 1002 are advertised and are purchasable by a consumer, and other interfaces 1018. For example, a tenant may, using a client device, request (service request 1034) one or more services provided by cloud infrastructure system 1002 using one or more of interfaces 1014, 1016, and 1018. For example, a tenant may access the online store, browse cloud services offered by cloud infrastructure system 1002, and place a subscription order for one or more services offered by cloud infrastructure system 1002 that the tenant wishes to subscribe to. The service request may include information identifying the tenant and one or more services that the tenant desires to subscribe to. For example, a tenant may place a subscription order for a chatbot related service offered by cloud infrastructure system 1002. As part of the order, the client may provide information identifying the input (e.g. utterances).

In certain aspects, such as the embodiment depicted in FIG. 10, cloud infrastructure system 1002 may comprise an order management subsystem (OMS) 1020 that is configured to process the new order. As part of this processing, OMS 1020 may be configured to: create an account for the tenant, if not done already; receive billing and/or accounting information from the tenant that is to be used for billing the tenant for providing the requested service to the tenant; verify the tenant information; upon verification, book the order for the tenant; and orchestrate various workflows to prepare the order for provisioning.

Once properly validated, OMS 1020 may then invoke the order provisioning subsystem (OPS) 1024 that is configured to provision resources for the order including processing, memory, and networking resources. The provisioning may include allocating resources for the order and configuring the resources to facilitate the service requested by the tenant order. The manner in which resources are provisioned for an order and the type of the provisioned resources may depend upon the type of cloud service that has been ordered by the tenant. For example, according to one workflow, OPS 1024 may be configured to determine the particular cloud service being requested and identify a number of pods that may have been pre-configured for that particular cloud service. The number of pods that are allocated for an order may depend upon the size/amount/level/scope of the requested service. For example, the number of pods to be allocated may be determined based upon the number of users to be supported by the service, the duration of time for which the service is being requested, and the like. The allocated pods may then be customized for the particular requesting tenant for providing the requested service.

Cloud infrastructure system 1002 may send a response or notification 1044 to the requesting tenant to indicate when the requested service is now ready for use. In some instances, information (e.g., a link) may be sent to the tenant that enables the tenant to start using and availing the benefits of the requested services.

Cloud infrastructure system 1002 may provide services to multiple tenants. For each tenant, cloud infrastructure system 1002 is responsible for managing information related to one or more subscription orders received from the tenant, maintaining tenant data related to the orders, and providing the requested services to the tenant or clients of the tenant. Cloud infrastructure system 1002 may also collect usage statistics regarding a tenant's use of subscribed services. For example, statistics may be collected for the amount of storage used, the amount of data transferred, the number of users, and the amount of system up time and system down time, and the like. This usage information may be used to bill the tenant. Billing may be done, for example, on a monthly cycle.

Cloud infrastructure system 1002 may provide services to multiple tenants in parallel. Cloud infrastructure system 1002 may store information for these tenants, including possibly proprietary information. In certain aspects, cloud infrastructure system 1002 comprises an identity management subsystem (IMS) 1028 that is configured to manage tenant's information and provide the separation of the managed information such that information related to one tenant is not accessible by another tenant. IMS 1028 may be configured to provide various security-related services such as identity services, such as information access management, authentication and authorization services, services for managing tenant identities and roles and related capabilities, and the like.

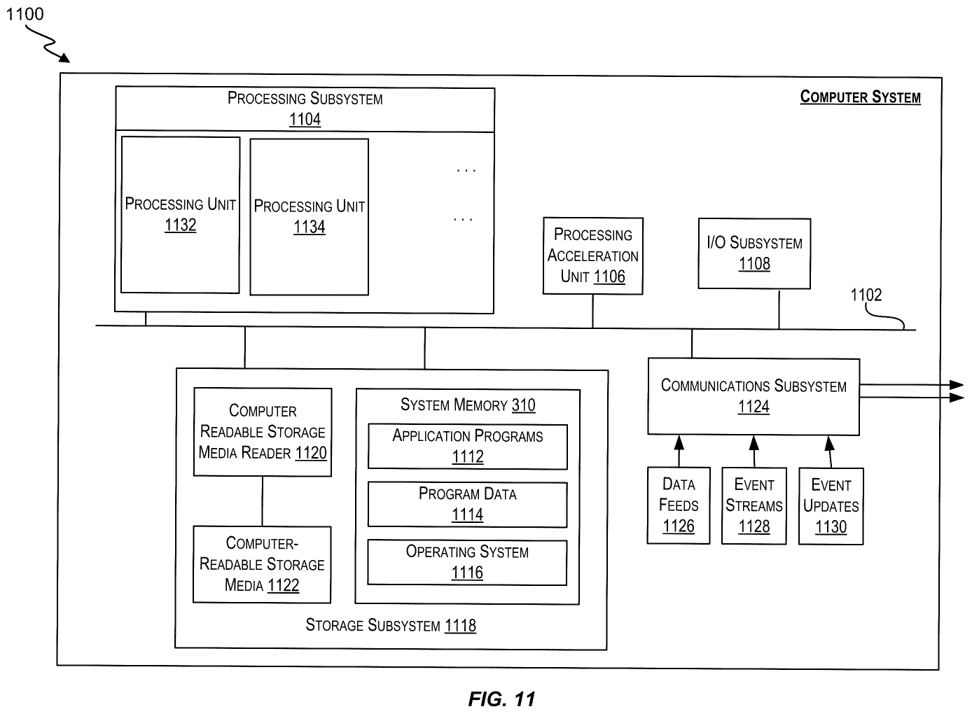

FIG. 11 illustrates an exemplary computer system 1100 that may be used to implement certain aspects. As shown in FIG. 11, computer system 1100 includes various subsystems including a processing subsystem 1104 that communicates with a number of other subsystems via a bus subsystem 1102. These other subsystems may include a processing acceleration unit 1106, an I/O subsystem 1108, a storage subsystem 1118, and a communications subsystem 1124. Storage subsystem 1118 may include non-transitory computer-readable storage media including storage media 1122 and a system memory 1110.

Bus subsystem 1102 provides a mechanism for letting the various components and subsystems of computer system 1100 communicate with each other as intended. Although bus subsystem 1102 is shown schematically as a single bus, alternative aspects of the bus subsystem may utilize multiple buses. Bus subsystem 1102 may be any of several types of bus structures including a memory bus or memory controller, a peripheral bus, a local bus using any of a variety of bus architectures, and the like. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard, and the like.

Processing subsystem 1104 controls the operation of computer system 1100 and may comprise one or more processors, application specific integrated circuits (ASICs), or field programmable gate arrays (FPGAs). The processors may be single core or multicore processors. The processing resources of computer system 1100 can be organized into one or more processing units 1132, 1134, etc. A processing unit may include one or more processors, one or more cores from the same or different processors, a combination of cores and processors, or other combinations of cores and processors. In some aspects, processing subsystem 1104 can include one or more special purpose co-processors such as graphics processors, digital signal processors (DSPs), or the like. In some aspects, some or all of the processing units of processing subsystem 1104 can be implemented using customized circuits, such as application specific integrated circuits (ASICs), or field programmable gate arrays (FPGAs).

In some aspects, the processing units in processing subsystem 1104 can execute instructions stored in system memory 1110 or on computer readable storage media 1122. In various aspects, the processing units can execute a variety of programs or code instructions and can maintain multiple concurrently executing programs or processes. At any given time, some or all of the program code to be executed can be resident in system memory 1110 and/or on computer-readable storage media 1122 including potentially on one or more storage devices. Through suitable programming, processing subsystem 1104 can provide various functionalities described above. In instances where computer system 1100 is executing one or more virtual machines, one or more processing units may be allocated to each virtual machine.

In certain aspects, a processing acceleration unit 1106 may optionally be provided for performing customized processing or for off-loading some of the processing performed by processing subsystem 1104 so as to accelerate the overall processing performed by computer system 1100.

I/O subsystem 1108 may include devices and mechanisms for inputting information to computer system 1100 and/or for outputting information from or via computer system 1100. In general, use of the term input device is intended to include all possible types of devices and mechanisms for inputting information to computer system 1100. User interface input devices may include, for example, a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may also include motion sensing and/or gesture recognition devices such as the Meta Quest® controller, Microsoft Kinect® motion sensor, the Microsoft Xbox® 360 game controller, or devices that provide an interface for receiving input using gestures and spoken commands. User interface input devices may also include eye gesture recognition devices such as a blink detector that detects eye activity (e.g., “blinking” while taking pictures and/or making a menu selection) from users and transforms the eye gestures as inputs to an input device. Additionally, user interface input devices may include voice recognition sensing devices that enable users to interact with voice recognition systems (e.g., Siri® navigator or Amazon Alexa®) through voice commands.

Other examples of user interface input devices include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, QR code readers, barcode readers, 3D scanners, 3D printers, laser rangefinders, and eye gaze tracking devices. Additionally, user interface input devices may include, for example, medical imaging input devices such as computed tomography, magnetic resonance imaging, position emission tomography, and medical ultrasonography devices. User interface input devices may also include, for example, audio input devices such as MIDI keyboards, digital musical instruments, and the like.

In general, use of the term output device is intended to include all possible types of devices and mechanisms for outputting information from computer system 1100 to a user or other computer. User interface output devices may include a display subsystem, indicator lights, or non-visual displays such as audio output devices, etc. The display subsystem may be any device for outputting a digital picture. Example display devices include flat panel display devices such as those using a light emitting diode (LED) display, a liquid crystal display (LCD) or plasma display, a projection device, a touch screen, a desktop or laptop computer monitor, and the like. As another example, wearable display devices such as Meta Quest® or Microsoft HoloLens® may be mounted to the user for displaying information. User interface output devices may include, without limitation, a variety of display devices that visually convey text, graphics, and audio/video information such as monitors, printers, speakers, headphones, automotive navigation systems, plotters, voice output devices, and modems.

Storage subsystem 1118 provides a repository or data store for storing information and data that is used by computer system 1100. Storage subsystem 1118 provides a tangible non-transitory computer-readable storage medium for storing the basic programming and data constructs that provide the functionality of some aspects. Storage subsystem 1118 may store software (e.g., programs, code modules, instructions) that when executed by processing subsystem 1104 provides the functionality described above. The software may be executed by one or more processing units of processing subsystem 1104. Storage subsystem 1118 may also provide a repository for storing data used in accordance with the teachings of this disclosure.

Storage subsystem 1118 may include one or more non-transitory memory devices, including volatile and non-volatile memory devices. As shown in FIG. 11, storage subsystem 1118 includes a system memory 1110 and a computer-readable storage media 1122. System memory 1110 may include a number of memories including a volatile main random access memory (RAM) for storage of instructions and data during program execution and a non-volatile read only memory (ROM) or flash memory in which fixed instructions are stored. In some implementations, a basic input/output system (BIOS), containing the basic routines that help to transfer information between elements within computer system 1100, such as during start-up, may typically be stored in the ROM. The RAM typically contains data and/or program modules that are presently being operated and executed by processing subsystem 1104. In some implementations, system memory 1110 may include multiple different types of memory, such as static random access memory (SRAM), dynamic random access memory (DRAM), and the like.

By way of example, and not limitation, as depicted in FIG. 11, system memory 1110 may load application programs 1112 that are being executed, which may include various applications such as Web browsers, mid-tier applications, relational database management systems (RDBMS), etc., program data 1114, and an operating system 1116. By way of example, operating system 1116 may include various versions of Microsoft Windows®, Apple Macintosh®, and/or Linux® operating systems, a variety of commercially-available UNIX® or UNIX-like operating systems (including without limitation the variety of GNU/Linux operating systems, the Oracle Linux®, Google Chrome® OS, and the like) and/or mobile operating systems such as iOS, Windows® Phone, Android® OS, and others.

Computer-readable storage media 1122 may store programming and data constructs that provide the functionality of some aspects. Computer-readable media 1122 may provide storage of computer-readable instructions, data structures, program modules, and other data for computer system 1100. Software (programs, code modules, instructions) that, when executed by processing subsystem 1104 provides the functionality described above, may be stored in storage subsystem 1118. By way of example, computer-readable storage media 1122 may include non-volatile memory such as a hard disk drive, a magnetic disk drive, an optical disk drive such as a CD ROM, digital video disc (DVD), a Blu-Ray® disk, or other optical media. Computer-readable storage media 1122 may include, but is not limited to, Zip® drives, flash memory cards, universal serial bus (USB) flash drives, secure digital (SD) cards, DVD disks, digital video tape, and the like. Computer-readable storage media 1122 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, dynamic random access memory (DRAM)-based SSDs, magneto-resistive RAM (MRAM) SSDs, and hybrid SSDs that use a combination of DRAM and flash memory based SSDs.

In certain aspects, storage subsystem 1118 may also include a computer-readable storage media reader 1120 that can further be connected to computer-readable storage media 1122. Reader 1120 may receive and be configured to read data from a memory device such as a disk, a flash drive, etc.

In certain aspects, computer system 1100 may support virtualization technologies, including but not limited to virtualization of processing and memory resources. For example, computer system 1100 may provide support for executing one or more virtual machines. In certain aspects, computer system 1100 may execute a program such as a hypervisor that facilitated the configuring and managing of the virtual machines. Each virtual machine may be allocated memory, compute (e.g., processors, cores), I/O, and networking resources. Each virtual machine generally runs independently of the other virtual machines. A virtual machine typically runs its own operating system, which may be the same as or different from the operating systems executed by other virtual machines executed by computer system 1100. Accordingly, multiple operating systems may potentially be run concurrently by computer system 1100.

Communications subsystem 1124 provides an interface to other computer systems and networks. Communications subsystem 1124 serves as an interface for receiving data from and transmitting data to other systems from computer system 1100. For example, communications subsystem 1124 may enable computer system 1100 to establish a communication channel to one or more client devices via the Internet for receiving and sending information from and to the client devices. For example, the communications subsystem may be used to transmit a response to a user regarding the inquiry for a chatbot.

Communications subsystem 1124 may support both wired and/or wireless communication protocols. For example, in certain aspects, communications subsystem 1124 may include radio frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 802.XX family standards, or other mobile communication technologies, or any combination thereof), global positioning system (GPS) receiver components, and/or other components. In some aspects communications subsystem 1124 can provide wired network connectivity (e.g., Ethernet) in addition to or instead of a wireless interface.

Communications subsystem 1124 can receive and transmit data in various forms. For example, in some aspects, in addition to other forms, communications subsystem 1124 may receive input communications in the form of structured and/or unstructured data feeds 1126, event streams 1128, event updates 1130, and the like. For example, communications subsystem 1124 may be configured to receive (or send) data feeds 1126 in real-time from users of social media networks and/or other communication services such as Twitter® feeds, Facebook® updates, web feeds such as Rich Site Summary (RSS) feeds, and/or real-time updates from one or more third party information sources.

In certain aspects, communications subsystem 1124 may be configured to receive data in the form of continuous data streams, which may include event streams 1128 of real-time events and/or event updates 1130, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1124 may also be configured to communicate data from computer system 1100 to other computer systems or networks. The data may be communicated in various different forms such as structured and/or unstructured data feeds 1126, event streams 1128, event updates 1130, and the like to one or more databases that may be in communication with one or more streaming data source computers coupled to computer system 1100.

Computer system 1100 can be one of various types, including a handheld portable device (e.g., an iPhone® cellular phone, an iPad® computing tablet, a personal digital assistant (PDA)), a wearable device (e.g., a Meta Quest® head mounted display), a personal computer, a workstation, a mainframe, a kiosk, a server rack, or any other data processing system. Due to the ever-changing nature of computers and networks, the description of computer system 1100 depicted in FIG. 11 is intended only as a specific example. Many other configurations having more or fewer components than the system depicted in FIG. 11 are possible. Based on the disclosure and teachings provided herein, a person of ordinary skill in the art can appreciate other ways and/or methods to implement the various aspects.

Although specific aspects have been described, various modifications, alterations, alternative constructions, and equivalents are possible. Embodiments are not restricted to operation within certain specific data processing environments but are free to operate within a plurality of data processing environments. Additionally, although certain aspects have been described using a particular series of transactions and steps, it should be apparent to those skilled in the art that this is not intended to be limiting. Although some flowcharts describe operations as a sequential process, many of the operations can be performed in parallel or concurrently. In addition, the order of the operations may be rearranged. A process may have additional steps not included in the figure. Various features and aspects of the above-described aspects may be used individually or jointly.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation, such as by executing computer instructions or code, or processors or cores programmed to execute code or instructions stored on a non-transitory memory medium, or any combination thereof. Processes can communicate using a variety of techniques including but not limited to conventional techniques for inter-process communications, and different pairs of processes may use different techniques, or the same pair of processes may use different techniques at different times.

Specific details are given in this disclosure to provide a thorough understanding of the aspects. However, aspects may be practiced without these specific details. For example, well-known circuits, processes, algorithms, structures, and techniques have been shown without unnecessary detail in order to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specification and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

What is claimed is:

1. A computer-program product tangibly embodied in a non-transitory machine-readable storage medium, including instructions configured to cause one or more data processors to perform a set of actions including:
detecting an event trigger related to an initiation of a subject encounter in a healthcare provider domain;
generating, in real-time, one or more ADT (admission, discharge, transfer) messages associated with the event trigger via an interface of a healthcare provider of the healthcare provider domain, wherein the interface is configured to transmit the one or more ADT messages to a backend cell;
receiving, in real-time, the one or more ADT messages from the interface by the backend cell that is configured within a cloud service;
processing, via a frontend cell configured within the cloud service, the one or more ADT messages by assigning a master subject index to uniquely identify each subject across the healthcare provider domain;
identifying one or more external entities from the one or more ADT messages based on identifiers assigned by the frontend cell;
storing the one or more ADT messages in association with the master subject index and the identifiers to record an encounter lifecycle including the subject encounter and one or more related subject encounters;
dynamically selecting from the one or more ADT messages, via the frontend cell, based on subject-based data-sharing permissions that are configured by the healthcare provider for each of the one or more external entities; and
transmitting, in real-time, the selected one or more ADT messages to each of the one or more external entities via an interoperability interface configured within the frontend cell.

2. The computer-program product of claim 1, wherein the set of actions further includes:
receiving a request from an external entity of the one or more external entities, via the interoperability interface, to display the encounter lifecycle; and
displaying the encounter lifecycle to the external entity based on the subject-based data-sharing permissions via a graphical user interface.

3. The computer-program product of claim 1, wherein the set of actions further includes:
acknowledging or rejecting, via the backend cell, the one or more ADT messages received from the healthcare provider domain based on validity or compliance with predefined rules.

4. The computer-program product of claim 1, wherein the backend cell comprises a load balancer configured to distribute the processing of the one or more ADT messages across a plurality of application fleets to manage incoming traffic from the healthcare provider domain.

5. The computer-program product of claim 1, wherein the one or more ADT messages include notifications associated with one or more of subject encounters including:
admission, transfer, discharge, visit, end visit, register, pre-admission and update information.

6. The computer-program product of claim 1, wherein the one or more ADT messages comply with HL7 standard.

7. A computer-program product tangibly embodied in a non-transitory machine-readable storage medium, including instructions configured to cause one or more data processors to perform a set of actions including:
creating, via a console, a query endpoint that is associated with an interoperability interface, configured to transmit one or more requests from an external entity to access clinical information associated with a particular subject encounter from a healthcare provider domain;
creating a delivery endpoint, via the console, to receive the clinical information back to the external entity;
establishing a communication channel between the healthcare provider domain and the external entity by linking the query endpoint and the delivery endpoint, wherein the communication channel is configured for a subject associated with the particular subject encounter;
processing, via an engine configured in a cloud service, the one or more requests received through the communication channel, wherein the engine:
identifies the subject and a healthcare provider associated with the subject encounter holding the clinical information based on a master subject index (MSI) that uniquely identifies a subject across a healthcare provider domain; and
applies subject-specific data-sharing permissions to determine whether the clinical information is authorized to share with the external entity;
retrieving the clinical information from the identified healthcare provider based on the authorization;
generating a response including the clinical information in a standardized format via the engine; and
transmitting the response to the external entity via the communication channel.

8. The computer-program product of claim 7, wherein the set of actions further includes:
receiving, via the communication channel, real-time ADT feed associated with the subject based on triggering of a subject encounter that involves the identified healthcare provider and the external entity.

9. The computer-program product of claim 7, wherein the set of actions further includes:
receiving, via the communication channel, a new request from the external entity comprising access for additional clinical information associated with a particular encounter or changes in the subject-specific data-sharing permissions between the external entity and the healthcare provider;
updating configurations of components within the cloud service using a control plane API based on the new request;
retrieving, via the engine, the master subject index based on the updated configurations;
generating a new response comprising a C62 bundle including the additional clinical information; and
transmitting the new response to the external entity via the communication channel.

10. The computer-program product of claim 7, wherein the set of actions further includes:
creating a client application, via the console associated with the external entity, within a client identity domain, wherein the client application requests for an access token from the client identity domain;
inputting, via the console, identifiers associated with the client identity domain and the client application;
obtaining, based on the identifiers, the access token from the client identity domain that authenticates the client to access the clinical information; and
receiving the one or more requests, including the access token, from the client application via the query endpoint.

11. The computer-implemented method of claim 7, wherein the query endpoint is configured to receive the one or more requests concurrently from the external entity, with request rate limits applied via an API gateway.

12. The computer-implemented method of claim 7, wherein the set of actions further includes:
caching the clinical information temporarily at the engine to be accessed within a predetermined timeout period upon failure of fulfilling the requests.

13. The computer-implemented method of claim 7, wherein the external entity uses a service uniform resource locator (URL) as the query endpoint.

14. A computer-program product tangibly embodied in a non-transitory machine-readable
storage medium, including instructions configured to cause one or more data processors to perform a set of actions including:
detecting an event trigger related to an initiation of a subject encounter in a healthcare provider domain;
generating, in real-time, one or more ADT (admission, discharge, transfer) messages associated with the event trigger via an interface of a healthcare provider of the healthcare provider domain, wherein the interface is configured to transmit the one or more ADT messages to a backend cell;
receiving, in real-time, the one or more ADT messages from the interface by the backend cell that is configured within a cloud service;
processing, via a frontend cell configured within the cloud service, the one or more ADT messages by assigning a master subject index to uniquely identify each subject across the healthcare provider domain;
identifying one or more external entities from the one or more ADT messages based on identifiers assigned by the frontend cell;
storing the one or more ADT messages in association with the master subject index and the identifiers to record an encounter lifecycle including the subject encounter and one or more related subject encounters;
dynamically selecting from the one or more ADT messages, via the frontend cell, based on subject-based data-sharing permissions that are configured by the healthcare provider for each of the one or more external entities; and
transmitting, in real-time, the selected one or more ADT messages to each of the one or more external entities via an interoperability interface configured within the frontend cell.

15. The computer-implemented method of claim 14, further comprising:
receiving a request from an external entity of the one or more external entities, via the interoperability interface, to display the encounter lifecycle; and
displaying the encounter lifecycle to the external entity based on the subject-based data-sharing permissions via a graphical user interface.

16. The computer-implemented method of claim 13, further comprising:
acknowledging or rejecting, via the backend cell, the one or more ADT messages received from the healthcare provider domain based on validity or compliance with predefined rules.

17. A computer-implemented method comprising:
creating, via a console, a query endpoint that is associated with an interoperability interface, configured to transmit one or more requests from an external entity to access clinical information associated with a particular subject encounter from a healthcare provider domain;
creating a delivery endpoint, via the console, to receive the clinical information back to the external entity;
establishing a communication channel between the healthcare provider domain and the external entity by linking the query endpoint and the delivery endpoint, wherein the communication channel is configured for a subject associated with the particular subject encounter;
processing, via an engine configured in a cloud service, the one or more requests received through the communication channel, wherein the engine:
identifies the subject and a healthcare provider associated with the subject encounter holding the clinical information based on a master subject index (MSI) that uniquely identifies a subject across a healthcare provider domain; and
applies subject-specific data-sharing permissions to determine whether the clinical information is authorized to share with the external entity;
retrieving the clinical information from the identified healthcare provider based on the authorization;
generating a response including the clinical information in a standardized format via the engine; and
transmitting the response to the external entity via the communication channel.

18. The computer-implemented method of claim 17, further comprising:
receiving, via the communication channel, real-time ADT feed associated with the subject based on triggering of a subject encounter that involves the identified healthcare provider and the external entity.
